SEQUENCE LISTING (3 sequences)

SEQ ID NO: 1 - protein, 11 aa, Medicago truncatula
MASSSETSSTN

SEQ ID NO: 2 - protein, 32 aa, Arabidopsis thaliana
MASISTPFPISLHPKTVRSKPLKFRVLTRPIK

SEQ ID NO: 3 - protein, 17 aa, Artificial sequence
  Synthetic sequence N-terminal sequence, after removal of chloroplastic transit sequence, of amino acid sequence predicted by DNA sequence analysis of the clone pDEST14AOS5
MASGSETPDLTVATRTG

(19) United States
(12) Patent Application Publication
(10) Pub. No.: US 2010/0035311 A1
(43) Pub. Date: Feb. 11, 2010

(54) METHOD AND COMPOSITION FOR ENZYME STORAGE

(76) Inventors: Roderick Casey, Norfolk (GB); Richard Keith Hughes, Norfolk (GB)

Correspondence Address: MICHAEL BEST & FRIEDRICH LLP, ONE SOUTH PINCKNEY STREET, P O BOX 1806, MADISON, WI, 53701, US

(21) Appl. No.: 12/443,211

(22) PCT Filed: Oct. 16, 2007
(86) PCT No.: PCT/GB07/03954

§ 371 Date: Mar. 27, 2009

(30) Foreign Application Priority Data
Oct. 16, 2006 (GB) 0620494.5

(51) Int. Cl. C12P 7/00; C12N 9/02

(52) U.S. Cl. 435/132; 435/189

(57) ABSTRACT

We describe the detailed production, biochemical characterisation and storage of CYP74 enzymes, including CYP74C3, a recombinant plant cytochrome P450 enzyme with hydroperoxide lyase (HPL) activity from Medicago truncatula, and CYP74A1, an Allene Oxide Synthase (AOS), from Arabidopsis thaliana. Steady state kinetic parameters, substrate and product specificities, Rz, extinction coefficient, haem content, and new ligands are disclosed. We show, on the basis of gel filtration, sedimentation velocity (sedimentation coefficient distribution) and sedimentation equilibrium (molecular weight) analyses that the CYP74 enzymes have low enzyme activity as a detergent-free, water-soluble, monomer. The enzyme activity can be completely restored by reactivation with detergent micelles, but not detergent monomers. Corresponding changes in the spin state equilibrium, and probably coordination of the haem-iron, are novel for P450 enzymes and suggest that detergent micelles have a subtle effect on protein conformation, rather than substrate presentation, which is sufficient to improve substrate binding and turnover number by an order of magnitude. The kcat/Km of up to 1.6×108 M-1. s-1 is amongst the highest recorded, which is remarkable for an enzyme whose reaction mechanism involves the scission of a C—C bond. We carry out both kinetic and biophysical studies to demonstrate that these effects, and conclude that these result from the formation of a complex between a protein monomer and a single detergent micelle. Association with a detergent micelle rather than oligomeric state represents a new mechanism of activation for membrane-associated P450 enzymes. Highly concentrated and monodispersed samples of detergent-free CYP74C3 and CYP74A1 proteins may be well suited for the purposes of crystallisation and structural resolution of the first plant cytochrome P450 enzyme. We further provide methods and compositions for stable storage of the CYP74 enzymes, including but not limited to HPL and AOS.

### TECHNICAL FIELD

The present invention relates generally to methods and materials for use in making and stably storing CYP74 enzymes, including HPL and AOS enzymes, that heretofore have been difficult to store due to instability.

### BACKGROUND TO THE INVENTION

Cytochrome P450 enzymes are widespread in Nature, but members of the CYP74 sub-family of these enzymes, which are common in plants, have not been studied extensively. CYP74 enzymes are very different from other P450 enzymes (for example, CYP73 plant enzymes like cinnamate hydroxylases [1], or classical P450 enzymes of microbial [2] or mammalian [3] origin) in that they have an atypical reaction mechanism that requires neither oxygen nor an NADPH-reductase [4], and as a consequence have extraordinarily high turnover numbers. In this sense, they have more in common with non-classical mammalian P450 enzymes like thromboxane synthase [5].

Hydroperoxide lyase (HPL), or hemiacetal synthase [6], is a member of this CYP74 sub-family and has an important role in oxylipin metabolism, plant defence and the food industry [7,8]. The enzyme cleaves hydroperoxides, formed from the oxygenation of polyunsaturated fatty acids by the action of lipoxygenase (LOX), into an array of volatile and non-volatile products that have both antibacterial and organoleptic properties [7]. HPL has the same substrate specificity as another class of CYP74 enzyme, allene oxide synthase (AOS), which has been studied in much greater detail [9]. Unlike HPL, which cleaves hydroperoxides, AOS transforms hydroperoxides into unstable fatty acid epoxides; the mammalian equivalent of AOS is prostaglandin endoperoxide H synthase [10] but there is no known mammalian equivalent of HPL. The molecular mechanisms and primary determinants of this difference in specificity are unknown, primarily because there is no detailed structural and kinetic analysis of any homogeneously purified recombinant HPL. Structural and kinetic analysis of eukaryotic P450 enzymes is especially problematic because they are: (i) membrane- or microsomal-associated with a surface hydrophobic domain, usually located at the N-terminus of the protein [1,11,12]; (ii) highly insoluble in the absence of detergents, and/or form a heterogeneous mixture of higher oligomers [13,14]; (iii) often studied using crude extracts and not as homogeneous, well characterised, recombinant enzymes; (iv) poorly expressed in Escherichia coli and require engineering at their N-terminus to enhance water-solubility which means that the heterologously-expressed protein is not always biologically identical to the protein predicted from the cDNA; some N-terminal truncated P450 enzymes, for example, interacted differently with their reductase and exhibited changes in specific activity and product specificity; (v) relatively unstable and can not be stored long term; and (vi) of uncertain oligomeric status in relation to the active species in vitro. All HPLs are membrane-associated and require detergent for extraction and solubilisation. It has been difficult to resolve detergent and protein interactions, and consequently there is some disagreement about the oligomeric state of HPL purified from a number of higher plants, including guava fruit [15], bell pepper fruits [16,17], sunflower hypocotyls [18], apple fruits [19], tomato leaves [20] and fruits [21], soybean seedlings [22] and watermelon [23]. The enzyme has been reported to be either trimeric or tetrameric; the oligomeric state of various recombinant HPLs: CYP74B1 [24], CYP74B2 [25], CYP74B3 [26], CYP74B4 [13], CYP74B5 [15], CYP74C1 [27] and CYP74C2 [28], and most often the effects of detergent removal, have not been reported. The effects of detergent on increasing the activity of HPL are well documented [29] but the molecular mechanism responsible for this activation is unknown.

Noordermeer et al. (2001, FEBS Letters 489, 229-232) made spectroscopic studies on the active site of hydroperoxide lyase and tested the influence of detergents on its conformation.

However prior to the work of the present inventors, there were no examples of a detailed biochemical characterisation of any HPL. Homology modelling of HPL is difficult, due to very poor sequence identity with any other P450 enzymes whose structure has been solved, unrecognisable protein folds and the absence of structures for HPLs, or any plant cytochrome P450. The structure of the AOS domain of the coral AOS-LOX chimera has recently been solved and shown to be very similar to a catalase [9], but this is highly dissimilar to HPLs which are neither water soluble nor predicted to have a catalase-fold. The structures of a number of water soluble microbial P450 enzymes have been solved [3], however, those from eukaryotes, for example, CYP2B4 [30], CYP2C5 [3], CYP2C8 [31], CYP2C9 [32] or CYP3A4 [33] or CYP2A6 [34], tend to have surface features for membrane interactions, and, to obtain crystals, required modifications in protein sequence to improve their water solubility and oligomeric state in the absence of detergents. All these are also highly dissimilar to any HPL. Therefore, prior to the work of the present inventors, there remained a clear requirement for a detailed structural and kinetic analysis of HPL and of membrane-associated plant cytochrome P450 enzymes in general.

Hughes et al. (2006, Biochem. J. 395, 641-652) described the detailed biochemical characterization of CYP74C3, a recombinant plant cytochrome P450 enzyme with HPL (hydroperoxide lyase) activity from Medicago truncatula (barrel medic).

Hughes et al. (2006, FEBS Letters 580, 4189-4195) investigated the effects of detergent on the kinetics and oligomeric state of CYP74A1. However no disclosure was made of methods of stable storage of the enzyme.

CYP74 enzymes are important, for example, in the flavour industries, where contacting of the active enzyme with certain intermediates results in bio-conversion to desired flavors. Thus it can be seen that methods and compositions for stable storage of such enzymes would provide a contribution to the art.

### DISCLOSURE OF THE INVENTION

In the present specification, there is described a procedure to obtain mg quantities of a recombinant HPL from Medicago truncatula, herein classified as CYP74C3, and Allene Oxide Synthase (AOS), CYP74A1, from Arabidopsis thaliana, and carry out a detailed biochemical characterisation of CYP74C3 and CYP74A1, when isolated as a detergent-free proteins with low activity. Secondly, we report the results of comprehensive ligand binding studies. Third, we examine the effects of detergent or substrate on reactivating the detergent-free enzymes, and distinguish between effects of substrate presentation and protein conformation. Detergent and substrate-induced changes in the spin state equilibrium of the haem-iron, and their associated effects on haem coordination, are also reported. Comparisons are then made with other CYP74 and two mammalian membrane-associated P450 enzymes as a basis to help improve our understanding of the differences in the regulation of catalysis of CYP74 and classical P450 enzymes. The role of micellar-association and oligomeric status in defining CYP74C3 and CYP74A1 activity is discussed.

Finally, the present invention further provides methods, means and compositions for the stable storage of CYP74 enzymes, including HPL and AOS. The inventors found that by producing or isolating these enzymes in a detergent free environment and drying, for example by freeze drying, spin-vacuum drying, thin film spray drying or other means known in the art which respect the protein nature of the enzymes, extended storage life of the enzymes is achieved, upon solution reconstitution with an appropriate detergent (see in particular Examples 3 and 4 herein, and FIGS. 14-30).

Accordingly, it is an object of this invention to provide detergent-free compositions of CYP74 enzymes, including but not limited to HPL and AOS enzymes.

It is a further object of this invention to provide methods for long-term, stable storage of CYP74 enzymes, including but not limited to HPL and AOS enzymes, comprising detergent-free dry compositions of the CYP74 enzymes.

It is another object of this invention to provide methods of using detergent free, dry-stored HPL and AOS enzymes.

Other objects and advantages of this invention will be apparent from a review of the complete disclosure and claims appended hereto.

### BRIEF DESCRIPTION OF THE DRAWINGS

FIG. 1. Constructs for expression of CYP74C3 variants in E. coli

(a) Expression of CYP74C3 in pDEST17HPL-F+7; (b) Expression of untagged CYP74C3 in pDEST14HPL-F+8 and (c) Expression of untagged CYP74C3 without the first 11 N-terminal amino acids in pDEST14HPL-F−8.

FIG. 2. UV/Visible spectrum and SDS-PAGE analysis of purified detergent-free CYP74C3 Spectrum of detergent-free CYP74C3 (10.5 μM) in 100 mM sodium phosphate buffer, pH 6.5 and SDS-PAGE analysis of the same preparation (52.5 pmol).

FIG. 3. Reactivation of detergent-free CYP74C3 with detergent

(a) Dose response curve for reactivation of detergent-free CYP74C3 with the detergent Emulphogene. Detergent-free CYP74C3 was incubated with an equal volume of 100 mM sodium phosphate buffer, pH 6.5 or containing detergent (0.01-10.24 mM Emulphogene) for 15 min on ice. Samples (1.2 pmol) were then assayed for HPL activity in the standard assay with 20 μM 13α-HPOT; (b) Plot of predicted micelle concentration versus activity for calculation of K_{d} for Emulphogene binding and (c) Fit of velocity-substrate curves to one-site saturation model in SigmaPlot for detergent-free CYP74C3 (1.0 pmol, ∘---∘), CYP74C3 purified in detergent-buffer (0.5 pmol, ∘—∘) and detergent-free CYP74C3 after reactivation with 5 mM Emulphogene (0.5 pmol, ▾—▾). The CMC of Emulphogene at 0.125 mM is shown. Error bars are the means±standard errors of at least six determinations on two different enzyme preparations.

FIG. 4. Ligand-binding studies of detergent-free CYP74C3

(a) UV/Visible spectra of ligand-bound forms of oxidised native CYP74C3 (2 μM, [symbol] ); KCN (100 mM, ——); imidazole (50 mM, ---------); DMPhP (250 mM, — —). The spectrum of dithionite (50 mM) reduced CYP74C3 (2 μM) before () and after (——) reaction with CO (˜1 mM) are also shown. The increase in absorbance below 400 nm is due to the large excess of sodium dithionite used; (b) Difference spectrum for CO-binding to the dithionite-reduced enzyme. (c) Stopped-flow diode array spectral changes over a period of 200 s for cyanide (100 mM) binding to native oxidised CYP74C3 (2 μM). The reaction is biphasic with fits to two exponential functions giving k₁=7.9±0.3×10⁻² s⁻¹ (amplitude 0.045) and k₂=1.3±0.1×10⁻² s⁻¹ (amplitude 0.13). An isosbestic point at 419 nm was observed for the second phase. All spectra were recorded at pH 6.5 in 100 mM sodium phosphate buffer, pH 6.5 at 25° C., 1 cm path length.

FIG. 5. Gel filtration analysis of purified detergent-free CYP74C3

Purified CYP74C3 (2 ml, 5.6 mg) was loaded on a Superdex 75 26/60 column equilibrated and eluted at 1 ml/min with 100 mM sodium phosphate buffer, pH 6.5. Peak positions for species with Mᵣ corresponding to the size of a protein monomer and dimer are shown.

FIG. 6. Gel filtration analysis of purified CYP74C3 in detergent-buffer

Purified CYP74C3 (0.5 ml, 5 mg) in detergent-buffer was loaded on a Superdex 200 16/60 column equilibrated and eluted in detergent-buffer at 0.5 ml/min. Fractions were monitored at A₂₈₀ ( [symbol] ) and for CYP74C3 activity (------). Peak positions for species with Mᵣ corresponding to the size of different oligomers are shown.

FIG. 7. UV/Visible spectrum and SDS-PAGE analysis of purified detergent-free CYP74A1 monomer. Spectrum of detergent-free CYP74A1 (8.2 μM) in 100 mM sodium phosphate buffer, pH 6.5. Inset, SDS-PAGE analysis of the detergent-free preparation (2.5 pmol); the molecular weight (in kDa) of the protein markers is indicated.

FIG. 8. HPLC/MS analysis of CYP74A1 product specificity. Products were detected by LC/MS after reaction of (a) 9-HPOTE (584 μM) or (b) 9-HPODE (575 μM) with CYP74A1 (40 pmol) reactivated with 50 μM Emulphogene micelle in 0.1 M sodium phosphate buffer, pH 6.5 for 30 min at 37° C. The extracted ion chromatograms of the ketols (m/z=309/311, —) and of the cyclopentenone (m/z=291/293, ----) are shown. Identical products were detected using detergent-free CYP74A1 (data not shown).

FIG. 9. Reactivation of detergent-free CYP74A1 with detergent. Dose response curve for reactivation of detergent-free CYP74A1 with the detergent Emulphogene. Detergent-free CYP74A1 was incubated with an equal volume of 100 mM sodium phosphate buffer, pH 6.5 containing Emulphogene (0-360 μM micelle concentration) for 15 min on ice. Samples (0.63 pmol) were then assayed for AOS activity with 20 μM 13-HPOTE (----). The c.m.c. of Emulphogene at 1.25 μM is shown. Error bars are the means±standard errors of at least six determinations on two different enzyme preparations. The data were fitted to a one site saturation model in Sigmaplot (—) to estimate the K_{d} Emulphogene of 10.7±1.7 μM.

FIG. 10. Gel filtration analysis of CYP74A1. Purified CYP74A1 (0.5 ml, 200 μM) in 100 mM sodium phosphate buffer, pH 6.5 (—) or detergent-buffer containing 15.6 μM Emulphogene (••••) was loaded on a calibrated Superdex 200 16/60 column equilibrated and eluted at 1 ml/min with the same buffers. Peak positions for species with molecular mass corresponding to the size of a protein monomer, dimer, trimer and tetramer are shown. The activities of CYP74A1 purified in detergent-free sodium phosphate buffer (—) and detergent-buffer (∘---∘) are shown.

FIG. 11. Stabilities of freeze-dried enzyme according to this invention over a period of ten weeks. The upper line corresponds to CYP74C3 (HPLF) and the lower line corresponds to CYP74A1 (AOS).

FIG. 12. Stability of detergent-free freeze dried CYP74C3 and CYP74A1. Black diamonds represent CYP74C3 and grey squares represent CYP74A1.

FIG. 13. Instability of detergent-free solutions of CYP74C3 and CYP74A1 stored at 4 degrees Centigrade. Black diamonds represent CYP74C3 and grey squares represent CYP74A1.

FIG. 14. Instability of detergent containing solutions of CYP74C3 and CYP74A1 stored at 4 degrees Centigrade. Black diamonds represent CYP74C3 and grey squares represent CYP74A1.

FIG. 15. Instability of detergent-free solutions of CYP74C3 and CYP74A1 stored at −20 degrees Centigrade with 20% glycerol Black diamonds represent CYP74C3 and grey squares represent CYP74A1.

FIG. 16. Instability of detergent containing solutions of CYP74C3 and CYP74A1 stored at −20 degrees Centigrade with 20% glycerol. Black diamonds represent CYP74C3 and grey squares represent CYP74A1.

### DETAILED DESCRIPTION OF THE PREFERRED EMBODIMENTS

### Abbreviations

AOS, allene oxide synthase; BI, benzimidazole; CMC, critical micelle concentration; CYP74, cytochrome P450 subfamily 74; DMPhP, dimethylphenylphosphine; Emulphogene, polyoxyethylene 10 tridecyl ether; HPL, hydroperoxide lyase; HPOD, hydroperoxyoctadecadienoic acid; HPOT, hydroperoxyoctadecatrienoic acid; IMAC, immobilised metal affinity chromatography; ISD, In Source Decay; KCN, potassium cyanide; KCNS, potassium thiocyanate; LOX, lipoxygenase; P450, cytochrome P450; R_{z}, Reinheitzahl or purity index.

### CYP74C3

CYP74C3 from Medicago truncatula is a cytochrome P450 enzyme with HPL activity. CYP74C3 exhibited dual specificity turning over both 9- and 13-hydroperoxides and in this respect it is very similar to CYP74C1 [27] and CYP74C2 [28]. The R_{z} of 1.3 and molar extinction coefficient at 391 nm of 122000 M⁻¹. cm⁻¹ are the first reported for any HPL. The R_{z} is identical to that of coral AOS [47]. The molar extinction coefficient at 391 nm is also very similar to that reported for coral AOS (at 406 nm) of 100000 M⁻¹. cm⁻¹ [47] and for flax AOS (at 392 nm) of 140000 M⁻¹. cm^{-1 [48}]. It is not possible to make comparisons with the R_{z} of other purified HPLs because the published spectra show only absorbance above 300 nm [13,21,28]; an extinction coefficient of 39000 M⁻¹. cm⁻¹ has been calculated for CYP74B3 [21], but the purity of the enzyme preparation and haem content were not reported. UV/Visible and EPR spectroscopy of oxidised CYP74C3 both indicated that the protein was typical of a thiolate-ligated pentacoordinate, ferric high spin haem iron-containing enzyme, with the sixth position accessible for ligand binding [see 48]; some low spin haem iron was also present. The g values recorded for the resting enzyme were very similar to those reported for CYP74B1 (purified without detergent removal) at 8.05, 3.5 and 1.65 (high spin haem iron) and 2.38, 2.24 and 1.92 (low spin haem iron) [24]. Features in the UV/Visible spectrum of detergent-free CYP74C3 were very similar to flax AOS (Soret band at 392 nm) [48], but quite different to that of detergent-free CYP74B4 (Soret band at 420 nm) [49]; the latter was attributed to the major presence of low spin haem iron. The Soret band at 406 nm for coral AOS [47] was, like CYP74C3, attributed to a major presence of high spin haem iron. The positions of the α and β bands at 545 and 508 nm respectively in CYP74C3 are very different to those observed for most classical and non-classical P450 enzymes. For example, they are at 569 and 535 nm in CYP2B4 [49] and at 567 and 535 nm in thromboxane synthase [5]. However, they are more similar to those at 540 and 512 nm in flax AOS [48], and at 534 and 500 nm in coral AOS [47], both of which are CYP74 enzymes that were soluble in the absence of detergent.

Ligand-binding studies confirm that CYP74C3 is a cytochrome P450 enzyme. Previously, only dithionite reduction [13,18,21,24], CO [18,21,28] and NO [24] binding have been reported for a HPL. Imidazole, cyanide, dioxygen, thiazole, azide, pyridine, thiocyanate and benzimidazole have now all been shown to bind to CYP74C3. A detailed kinetic analysis was beyond the scope of the current investigation, but the resulting spin states and UV/Visible spectra have been determined and compared to other CYP74 enzymes and selected classical P450 enzymes. These are summarised in Table 2. The UV/Visible spectra of the cyanide and imidazole complexes of CYP74C3 compare favourably with those of CYP2B4 [50], CYP1A2 [50], CYP74B1 [24] and coral AOS [47]. The biphasic kinetics for cyanide binding are different to the monophasic kinetics reported with this ligand for thromboxane synthase [5], but the kinetics may be pH dependent due to the anticipated effects of the changes in protonation state of these ligands. The rapid-scan spectra for cyanide binding and stopped-flow time course for imidazole do demonstrate the relatively slow binding of ligands characteristic of similar P450 enzymes [5] rather than the rapid binding associated with other haem proteins such as globins and peroxidases [51]. Features in the DMPhP and BI complexes of CYP2B4 [50] and CYP1A2 [50] and the azide complex of coral AOS [47] are also very similar to those of CYP74C3.

Detergent micelles increased the k_{cat} of CYP74C3 by up to an order of magnitude through improvements in substrate binding. The very tight binding of 13α-HPOT with a Kₘ of only 3.3 μM, is amongst the lowest reported for any HPL. The very high turnover number of up to 657 s⁻¹ is the first reported for a HPL and is typical of a CYP74 enzyme, although somewhat lower than 4700 or 1200 s⁻¹, which were observed for flax AOS [48] and CYP74A2 [10] respectively, but this probably reflects differences in the reaction mechanisms of HPL and AOS. The k_{cat}/Kₘ of up to 1.6×10⁸ M⁻¹. s⁻¹ demonstrates that CYP74C3 is an extremely efficient catalyst matching that of carbonic anhydrase I [52]. This is particularly remarkable for an enzyme whose reaction mechanism involves the scission of a C—C bond in a relatively large (18C) fatty acid hydroperoxide substrate. Micelle-induced changes in turnover number were associated with subtle changes in both the Soret and visible regions of the spectrum of CYP74C3; the development of a shoulder at 420 nm and a new feature at 568 nm upon addition of detergent micelles to detergent-free CYP74C3 suggested a shift in equilibrium towards low spin haem iron, which was confirmed by EPR spectroscopy. The UV/Visible spectrum of CYP74C3, CYP74B1 [24] and CYP74B3 [21], purified without detergent removal, were very similar to one another with major Soret bands at 390-393 nm, characteristic of high spin haem iron. The UV/Visible spectrum of CYP74B4 purified without detergent removal also had a Soret band at 390 nm, typical of high spin haem iron [13], but it has been proposed that this was not detected under EPR conditions due to a temperature dependency of the signals [49], or by other workers [24] to imidazole contamination. The effects on the UV/Visible spectrum of adding detergent micelles to detergent-free CYP74C3 were very similar to those observed for CYP1A2 [53] or CYP2B4 [54] (see Table 2). In the presence of 10 mM n-octyl glucoside, a complete loss of catalytic activity and a shift to low spin haem iron of CYP2B4 with reductase was observed due to disaggregation of the active pentamer or hexamer into inactive monomers [14]. However, the effects of Emulphogene on CYP2B4 (and CYP1A2) activity appear to be more contradictory [55], with reports of both loss of, and increases in catalytic activity, due to the formation of monomers or dimers respectively. Certainly for the effects of Emulphogene on CYP74C3 in the present work, there was a positive correlation between the proportion of low spin haem iron (determined by both UV/Visible and EPR spectroscopy) and turnover number. Our observations are not the first to demonstrate the effect of detergent micelles on improving the catalytic activity of CYP74 enzymes [29, 48], but they are the first to explore the relationship between turnover number and oligomeric state for this class of P450 enzyme. Previously, it has been only speculated that the effect of detergent or high salt on increasing catalytic activity of a HPL was a result of some conformational change in the protein [29]. To our knowledge, no studies in this regard have been carried out for an AOS.

CYP74C3 required extraction and purification in the presence of detergent, but unlike other HPLs, it remained soluble and active as a monomer when both detergent and salt were removed. The protein was almost entirely monomeric at concentrations from 0.5 to 15 mg/ml over a wide range of pH and ionic strengths. In the present work, the most active CYP74C3 oligomer in the presence of detergent may have been a protein dimer, which is a new observation. This contrasts with other reports where HPL purified from higher plants was either a trimer or a tetramer [15-23]. In those instances where gel filtration was carried out in the presence of detergent and aggregation was prevented, the higher Mᵣ estimations may have been due to the formation of HPL complexes with the large (Mᵣ 90 kDa) detergent micelles of Triton X-100 that was used at concentrations well above the CMC (0.24 mM) of this detergent [40]. These HPLs may also have been considerably more hydrophobic than CYP74C3, which might explain their insolubility in the absence of detergent. In the present work, since Emulphogene was used at a concentration of 1.56 mM that is also well above the CMC of this detergent (0.125 mM), micelles of average Mᵣ 62.1 kDa would have formed under the conditions used for gel filtration. The identification of a CYP74C3 monomer and no dimer under detergent-free conditions was perhaps not surprising because any protein-bound detergent remaining would have been at concentrations well below the CMC, and the detergent would have been entirely monomeric. Any complex formation with protein and Emulphogene micelle would increase the Mᵣ from 56.8 to 118.9 kDa, but no protein of this size or activity at this peak position in gel filtration under detergent-free conditions was detected. In contrast, in the presence of detergent-buffer, the Mᵣ of the most active species was the size of a protein dimer, or of a complex between a protein monomer and an Emulphogene-micelle. The conclusion of dimer formation for CYP2B4 in the presence of detergent [55] was based on the results of gel-filtration in the presence of Emulphogene, but like CYP74C3, may have corresponded to a protein monomer-micelle complex, rather than a protein dimer.

Regardless of the method of purification, CYP74C3 under the conditions of the activity assay was almost entirely monomeric. Nevertheless, the k_{cat} of the detergent-free monomer with the preferred substrate was only 10% of the activity of the same enzyme in detergent-buffer. It may be suggested that the low level of activity in the detergent-free preparation was due to contamination with a small amount of more active higher oligomer. However, clean separation of the monomer from the dimer by gel filtration on Superdex 75, and the presence of enzyme activity in only the fraction corresponding to the size of the monomer, suggests that this was unlikely. Under detergent-free conditions, the 10% residual activity more likely resulted from CYP74C3 monomers, which may or may not have been contaminated with detergent monomers that coated hydrophobic patches on the surface of the protein. We propose that detergent-free monomers exhibited reduced substrate binding through a conformational change that was induced by release from a micellar or membrane environment. Complete reactivation of detergent-free CYP74C3 with the detergent Emulphogene was possible only at concentrations of at least 26-fold higher than the CMC, which suggested that detergent micelles (or the simulation of a membranous environment) and not monomers were required to maintain the most active conformation of CYP74C3. This evidence together with the observation that the smallest and most active protein species detected by gel-filtration in the presence of Emulphogene corresponded to the size of a monomer-micelle, suggested that an association between a protein monomer and a detergent micelle was the most active conformation for CYP74C3 and not a protein dimer. Further evidence for the lack of protein dimer formation comes from the absence in concentrated detergent-free CYP74C3 preparations before and after reactivation with Emulphogene of EPR signals corresponding to haem-haem interactions, and of protein sedimenting with this M_{w} in the analytical ultracentrifuge. The little protein dimer that was detected under detergent-free conditions in the analytical ultracentrifuge was shown not to be in reversible equilibrium with protein monomer. Improvements in the activity and binding kinetics for CYP74C3 purified in detergent-buffer were unexpected because the concentration of detergent present in the activity assay would have been well below the CMC. This must suggest that the interaction between detergent micelle and protein monomer was stable over the very short time course of the activity assay (20 s); the low K_{d} determined for micelle-binding of only 6.9 μM certainly indicated that this association was very tight, indeed, it was tighter than for the preferred substrate (Kₘ 20.9 μM).

13α-HPOT is only sparingly soluble in aqueous solution so it is important to distinguish between the substrate being presented from aqueous solution and from a micelle or membrane. It may be suggested that detergent micelles, or the formation of mixed substrate and detergent micelles, could facilitate substrate binding without any requirement for a change in protein conformation. However, only monomers of 13α-HPOT would be small enough to enter the active site of the protein and 13α-HPOT was entirely monomeric and did not form any micelles under our experimental conditions, even in the presence of Emulphogene micelles. This suggested that reactivation of detergent-free CYP74C3 (and the observed changes in spin state of the haem-iron) required either detergent-micelles or substrate monomers, and that substrate presentation could not account for CYP74C3 reactivation.

Without wishing to be bound by theory or mechanism, we hypothesise that in E coli, and most likely in planta, CYP74C3 is a peripheral membrane protein with small patches of hydrophobic surface residues that promote a tight association with membranes, most likely as monomers. Solubilisation with detergents releases the monomers from the membranes and allows them to self-associate to form a mixture of monomer-micelles and higher oligomers through hydrophobic interaction. The nature of the hydrophobic domain of CYP74C3 that is necessary for membrane association in CYP74C3 is unknown. The N-terminal sequence of P450 enzymes has been reported to be a membrane-binding domain [12], but the N-terminal sequence of CYP74C3 is not particularly hydrophobic and evidence from the present work suggests that it has no role in membrane-binding. The structure of the engineered AOS domain from an AOS-LOX fusion chimera was recently crystallised as a protein dimer [9] but this bears no similarity to CYP74C3. The present work would suggest that the association between a protein monomer and a single detergent micelle (or perhaps a phospholipid in a membrane), and not oligomeric state, regulates the catalytic activity of CYP74C3. This represents a new mechanism for a membrane-associated P450 enzyme and may be a distinguishing feature of CYP74 enzymes that are distinct from classical P450 enzymes that require association with a reductase in order to carry out their full range of biological activities.

Unlike other plant cytochrome P450 enzymes described so far, CYP74C3 is a natural variant that does not require protein engineering to improve water solubility. The detailed understanding of the physical and biochemical properties of CYP74C3 described in the present work has provided valuable information towards our understanding of the differences in reaction mechanism of CYP74 and more typical P450 enzymes and the first crystal or NMR solution structure for an HPL or other membrane-associated plant cytochrome P450 enzyme. The subsequent information that would be forthcoming on the primary determinants of substrate and product specificities, and an identification of the hydrophobic domain responsible for membrane-association, are likely to be key requirements for the overall aim of manipulating oxylipin metabolism in planta, which is challenging, particularly in the model plant Arabidopsis.

### CYP74A1

We have investigated the effects of detergent on the kinetics and oligomeric state of allene oxide synthase (AOS) from Arabidopsis thaliana (CYP74A1). We show that detergent-free CYP74A1 is monomeric and highly water soluble with dual specificity, but has relatively low activity. Detergent micelles promote a 48-fold increase in k_{cat}/Kₘ (to 5.9×10⁷ M⁻¹.s⁻¹) with concomitant changes in the spin state equilibrium of the haem-iron due to the binding of a single detergent micelle to the protein monomer, which is atypical of P450 enzymes. This mechanism is shown to be an important determinant of the substrate specificity of CYP74A1. CYP74A1 may be suited for structural resolution of the first plant cytochrome P450 and its 9-AOS activity and behaviour in vitro has implications for its role in planta.

Allene oxide synthase (AOS) from Arabidopsis thaliana (CYP74A1) [1′] is a member of the CYP74 sub-family of cytochrome P450 enzymes and has an important role in oxylipin metabolism and signalling in plant defence [2′]. CYP74 enzymes are non-classical P450 enzymes in that they have an atypical reaction mechanism that requires neither oxygen nor a NADPH-reductase [3′] and consequently have extraordinarily high catalytic centre activities. AOS transforms fatty acid hydroperoxides, formed from the oxygenation of polyunsaturated fatty acids by the action of lipoxygenase (LOX), into unstable fatty acid epoxides [4′]; the mammalian equivalent of AOS is prostaglandin endoperoxide H synthase [5]. CYP74A1 has been classified from phylogenetic analyses and in vitro enzyme assays, using recombinant protein expressed in Escherichia coli, as a 13-AOS [1′,2′]. Structural and kinetic analysis of eukaryotic P450 enzymes is problematic because they are usually membrane- or microsomal-associated, and are of uncertain oligomeric status in relation to the active species in vivo. All AOSs purified from plants are membrane-associated and require detergent for extraction and solubilisation. With one exception, that of AOS purified from flax seed [6′], the oligomeric state of AOS purified from a number of higher plants, including guayule [5′,7′] and corn [8′], and of recombinant AOSs from barley [9′] and tomato [10′, 11′] has not been reported, because it has proved difficult to resolve the detergent and protein interactions. Gel filtration analysis of AOS purified from flax seed in the presence of detergent confirmed that the protein remained as a monomer of molecular mass 55 kDa [6′], which suggested that there was no association with detergent micelles and that the protein was entirely water-soluble. In the same work, however, it was reported that the specific activity of the enzyme was enhanced 2-3 fold by detergent [6′], but the molecular mechanism responsible for this activation is unknown. The effects of removing detergent (or adding detergent to detergent-free protein) on catalytic activity and oligomerisation have not been reported for any AOS.

There is a clear requirement for a detailed characterisation of a plant AOS, particularly in relation to its behaviour in the presence and absence of detergent micelles. Homology modelling of plant AOSs is difficult, due to very poor sequence identity with any other P450 enzymes, so the realisation of what would be the first crystal structure for a plant cytochrome P450 may be dependent on information relating to the behaviour of AOS protein in solution, and the factors affecting its catalytic activity, oligomeric state and monodispersity. The structure of an AOS from coral has recently been solved as a protein dimer [12′], but this is the water-soluble N-terminal domain of a naturally occurring AOS-LOX fusion protein [13′]. It is not a CYP74 enzyme, has a catalase-like fold, and bears no similarity to any plant AOS. The use of an in vitro system to study AOS in the presence of detergent micelles (akin to a membrane associated state) and the absence of detergent micelles (akin to a free cytosolic state) is also relevant to assess the biological activity of AOS in planta, since the localisation of AOS and other enzymes, inhibitors, substrates and products of oxylipin metabolism are important determinants of the oxylipin profile of a plant [2′,14′].

We describe a procedure to obtain mg quantities of a homogeneously purified detergent-free and water soluble recombinant AOS from Arabidopsis thaliana (CYP74A1) with relatively low catalytic activity and report its kinetic parameters and other biochemical properties, including oligomeric state. We also describe the effects of detergent on the reactivation kinetics of the detergent-free enzyme, and the associated effects on oligomeric state, spin state equilibrium of the haem-iron and substrate specificity. Finally, we propose a mechanism for the reactivation, discuss the relationships between micelle-association and oligomeric status in defining the activity of CYP74A1 and compare them to those recently described for CYP74C3 [15′].

In different aspects, the present invention includes:
A detergent free composition comprising an isolated, optionally recombinant, CYP74 enzyme (such as HPL and/or AOS).
Such a composition which comprises CYP74C3, CYP74A1, or both.
Such a composition which exhibits HPL activity, AOS activity, or both.

### Stable Storage

In this patent disclosure, we provide detergent free compositions of CYP74 enzymes. We also demonstrate, surprisingly, that the detergent free enzymes may be dried and stored for extensive periods of time, at least 15 weeks, in the cold or at room temperature, and that upon reconstitution in an appropriate detergent containing solution, essentially 100% of enzyme activity is retained, whereas storage of detergent free or detergent containing solutions of the CYP74 enzymes, at 4 or -20 degrees Centigrade, results in rapid fall off of enzyme activity.

Preferably the invention provides a dry composition which comprises an isolated HPL or AOS enzyme in the absence of appreciable quantities of detergent which, when hydrated and contacted with an effective amount of detergent, exhibits hydrogen peroxide lyase activity or AOS activity, or both.

Other aspects of the invention are defined in the claims appended hereto. Thus the invention further provides a method for producing and optionally storing a stable preparation of an isolated (e.g. recombinant) CYP74 enzyme which comprises the steps of:

(i) providing the enzyme in a substantially detergent-free state, and
(ii) drying the detergent free enzyme,
(iii) optionally storing the preparation,
(iv) optionally solubilising the preparation in a detergent containing solution, or in a solution to which a detergent is added, such as to provide active CYP74 enzyme

“Isolated” means isolated and/or purified from its natural environment, in substantially pure or homogeneous form, or free or substantially free of other (non-CYP74 enzymes) of the species of origin.

The preparation will be “substantially free of detergent” by which is meant comprising than 0.1, preferably less than 0.05, 0.01, 0.05, 0.005, 0.001%. For example detergent may be removed by extensive washing of the enzyme when bound to a chromatography column.

Drying may be by conventional means e.g. freeze drying, spin-vacuum drying, thin film spray drying.

Preferably the preparation is capable of being stored for at least 15 weeks while losing less than 25%, 20%, 15%, or 10% CYP74 enzyme activity, using a “standard activity assay” such as those described for the enzymes in the Examples herein. Alternatively it may be determined by analysis of the haem absorption in the Soret band as described below.

Preferably the loss of activity in the preparation is less than 50%, 40%, 30%, 20%, or 10% of the corresponding loss activity of the corresponding CYP74 enzyme when stored in the presence of detergent under the same conditions for 15 weeks. The comparison, for example, may use Emulphogene (0.3% w/v) or Triton X-100 (0.2% w/v).

The invention further provides a stable preparation of an isolated CYP74 enzyme in dry form and substantially free of detergent, which is capable of being stored for at least 15 weeks while losing less than 25%, 20%, 15%, or 10% CYP74 enzyme activity when subsequently solubilised in a detergent containing solution, or in a solution to which a detergent is added.

The invention further provides use of the same to convert hydroperoxy compounds to \acute{ω}-oxo acids, volatile aldehydes, or allene oxides.

The stable preparations may be provided in vials of standard amounts—for example in kits with instructions for use and storage. A vial may contain, for example, 1 to 100 mg, or any (preferably) integral number of mg between these values e.g. 5, 10, 25, 50.

### EXAMPLES

Having generally described this invention, including with respect to its best mode, the following examples are provided to extend the enablement and written description of this invention. The invention, however, should not be construed as being limited to the specifics of the examples provided, but rather, for this purpose, reference should be made to the appended claims and the equivalents thereof.

Any sub-titles herein are included for convenience only, and are not to be construed as limiting the disclosure in any way.

The disclosure of all references cited herein, inasmuch as it may be used by those skilled in the art to carry out the invention, is hereby specifically incorporated herein by cross-reference.

### Example 1

### Characterization of CYP74C3

### Cloning and Expression

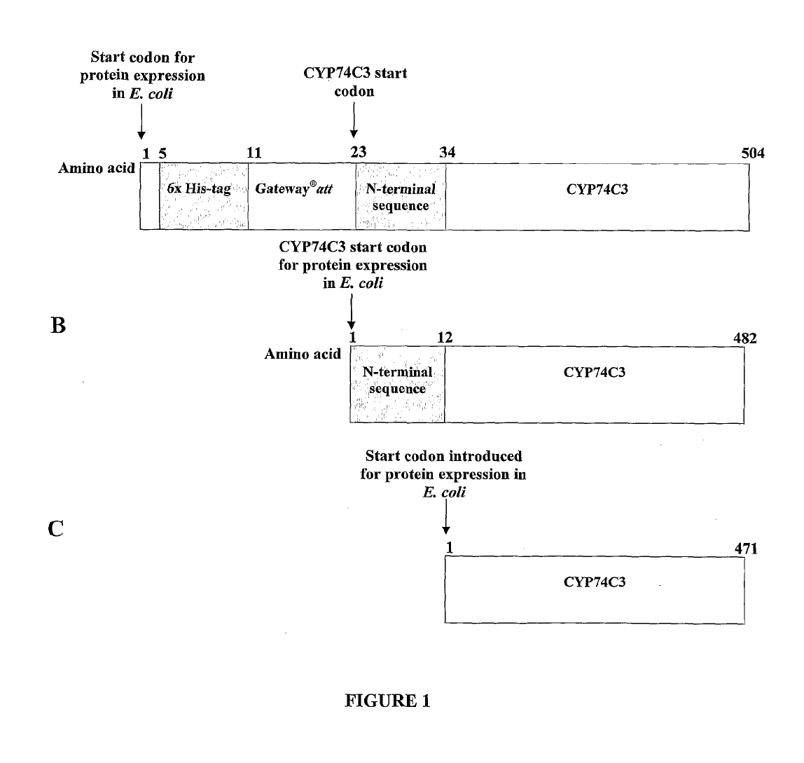

CYP74C3 cDNA (full-length EST clone obtained from the Samuel Roberts Noble Foundation, Ardmore, Okla., U.S.A.) was cloned into the destination vector pDEST17 using Gateway® technology (Invitrogen, Paisley, Scotland, U.K.) according to the manufacturer's instructions to give the plasmid pDEST17HPL-F+7. Expression of CYP74C3 protein in pDEST17HPL-F+7 occurred from the ATG start codon in pDEST17 (FIG. 1a), so the encoded protein had a 22 N-terminal amino acid extension sequence (including a 6×N-terminal histidine-tag and the peptide encoded by the Gateway® recombination att sequence). To determine whether this N-terminal sequence affected the oligomeric state [35] or kinetic properties of the expressed protein, the cDNA was also cloned into pDEST14 to give the plasmid pDEST14HPL-F+8 for expression of the untagged protein (FIG. 1b). To investigate the role, if any, of a putative membrane targeting or pro-enzymic N-terminal sequence of CYP74C3, the cDNA was also cloned in pDEST14 to give the plasmid pDEST14HPL-F−8 and expressed without the first 11 amino acids (MASSSETSSTN) at the N-terminus (FIG. 1c).

DNA sequence analysis of the clones pDEST14HPL-F+8 and pDEST14HPL-F−8 revealed that the predicted amino acid sequence of the protein expressed from both constructs was identical to that of the original cDNA clones. MALDI-ToF, Edman sequencing and ISD was used to confirm unambiguously that the sequence of the entire protein expressed from pDEST17HPL-F+7 was identical to that predicted from the original cDNA clone, except for the replacement of methionine at the start codon with an asparagine residue.

### Extraction and Purification of Histidine-Tagged CYP74C3

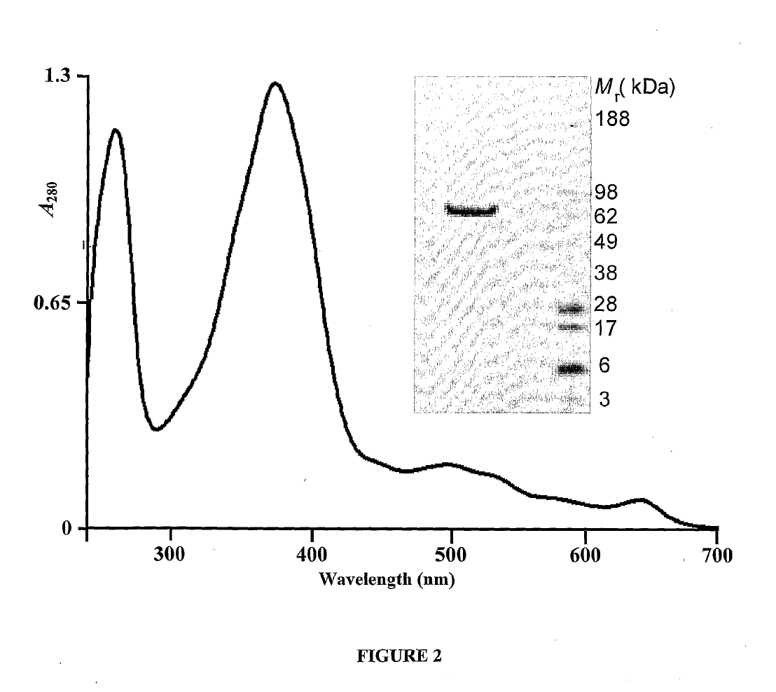

Cultures (20×2 L conical flasks each containing 1 L LB-G and 50 μg/ml ampicillin) of E. coli strain BL21 (DE3) transformed with expression plasmid were grown at 37° C. to A₆₀₀ 1.0-1.1 with shaking at 200 rpm, transferred to 21° C., and induced with IPTG (1 mM) for 20 h. Cells were harvested by centrifugation at 5,000 g and the pellets frozen at −80° C. Cell pellets were thawed and extracted at room temperature with 300 ml Bugbuster® (Novagen, Merck Biosciences Ltd., Beeston, Nottingham, U.K.) supplied in 50 mM Tris-HCl buffer, pH 8.0 containing 125 μl (3125 U) benzonase. Homogenates were then transferred to Oakridge (30 ml) centrifuge tubes, vortexed for 1 min, mixed gently by inversion for 30 min. All the following procedures were then carried out at 4° C. Homogenates were centrifuged at 28,000 g for 15 min and the supernatants were decanted on ice and loaded at 5 ml/min onto a 5 ml Hi-Trap IMAC column (Amersham Biosciences, GE Healthcare, Chalfont St. Giles, Buckinghamshire, U.K.) charged with cobalt chloride connected to an AKTA FPLC system (Amersham Biosciences). Unbound protein was eluted at 5 ml/min with approximately 250 ml of 50 mM KH₂PO₄/K₂HPO₄ (potassium phosphate) buffer, pH 7.6 containing 0.9 M NaCl, 50 mM glycine, 5% (v/v) glycerol and 1.56 mM Emulphogene (polyoxyethylene 10 tridecyl ether) (herein referred to as detergent-buffer). Excess detergent was then removed from the bound protein by washing with 125 ml detergent-buffer without Emulphogene (buffer B), followed by 125 ml 50 mM potassium phosphate buffer, pH 7.6 containing 0.15 M NaCl (buffer C). CYP74C3 was eluted at 5 ml/min with a linear gradient (50 ml, 10 min) from 0-40 mM histidine in buffer C. For purification of CYP74C3 in detergent-buffer, excess detergent was not removed with buffer B and the protein was eluted with a linear gradient from 0-40 mM histidine in detergent-buffer. In either case, fractions eluting at >36 mM histidine were pooled and concentrated to approximately 2 ml using Amicon Ultra 10,000 molecular weight cut-off centrifugal filter devices (Millipore, Watford, Hertfordshire, U.K.). For detergent-free protein, the concentrate (2 ml) was then injected at 0.2 ml/min onto a Hi-load Superdex 75 26/60 gel filtration column (Amersham Biosciences) equilibrated with 100 mM NaH₂PO₄/100 mM Na₂HPO₄ (sodium phosphate) buffer, pH 6.5 and eluted with the same buffer at 2 ml/min. For protein in detergent-buffer, the concentrate (0.5 ml) was injected at 0.1 ml/min onto a Superdex 200 16/60 gel filtration column (Amersham Biosciences) equilibrated in detergent-buffer and eluted with detergent-buffer at 1 ml/min. Fractions with the highest R_{z} (Reinheitzahl; A₃₉₁/A₂₈₀) were pooled and concentrated to 10 mg/ml. The R_{z} of the final preparation (detergent-free) was 1.3, very similar to the enzyme eluted in detergent-buffer. Detergent-free enzyme was snap frozen in 100 μl aliquots in liquid nitrogen and stored indefinitely at −80° C.; enzyme in detergent-buffer was best stabilised in the short term (1 month maximum) in 50% (v/v) glycerol at −20° C. Extraction of soluble histidine-tagged CYP74C3 activity and binding of this activity to an IMAC (immobilised metal affinity chromatography) column required the presence of detergent, suggesting that CYP74C3 is associated with membranes in E. coli. This localisation was confirmed in previous ultracentrifugation studies under detergent-free conditions where activity was almost exclusively associated with a detergent-solubilised membrane-enriched fraction (data not shown). The purified detergent-free CYP74C3 preparation was homogeneous as judged by SDS-PAGE (FIG. 2). Optimisation of the purification using Co- instead of Ni-IMAC was essential to remove minor contamination by an E. coli peptidyl-prolyl cis/trans isomerase (Accession number PIR2: S46294) [46] that was clearly identified by MALDI-ToF analysis (data not shown). This protein had a subunit Mᵣ of 22 kDa (44 kDa dimer in solution) but ran anomalously in SDS-PAGE with an apparent subunit Mᵣ of approximately 30 kDa. Elution with histidine rather than imidazole was essential to produce an active enzyme with the characteristic UV/Visible spectrum shown in FIG. 2; the spectrum was not dependent on protein concentration. The protein had a Soret band at 391 nm, and major features at 508, 545 and 644 nm. Elution of the enzyme with imidazole produced a virtually inactive enzyme (data not shown). Analysis using the pyridine haemochrome method confirmed that the enzyme had a full complement of type b haem (0.93±0.16 mol/mol protein). The R_{z} of pure protein was 1.3. The extinction coefficient at 391 nm was calculated to be 102000 M⁻¹. cm⁻¹ and 141000 M⁻¹. cm⁻¹ using the Bradford and BCA assays respectively. The mean value of 122000 M⁻¹. cm⁻¹ was used to calculate CYP74C3 concentration. The UV/Visible spectrum shown in FIG. 2 was of the protein purified as a monomer (see later). In some circumstances after gel filtration, however, a small proportion of soluble aggregated protein was also observed, which eluted at the void volume and had the same purity (as judged by SDS-PAGE) but a lower R_{z} (approximately 1.1). The latter was presumably due to the higher absorbance at 280 nm relative to haem of the aggregates. The UV/Visible spectrum of CYP74C3 purified without removing the Emulphogene detergent had a very similar spectrum and purity to the detergent-free protein, as judged by SDS-PAGE, but the same R_{z} as the detergent-free enzyme (1.3) (data not shown). The purity of untagged CYP74C3 expressed from pDEST14HPLF+8 was estimated by SDS-PAGE to be approximately 80-85%.

### Untagged CYP74C3

Detergent-free untagged CYP74C3 was purified by hydrophobic interaction chromatography and gel filtration. Cell pellets from cultures were induced and extracted exactly as described for histidine-tagged CYP74C3, except the crude supernatant was diluted with an equal volume of 100 mM potassium phosphate buffer, pH 7.6 containing 2 M ammonium sulphate and loaded at 5 ml/min onto a 5 ml Hi-Trap Phenyl Sepharose FF (low sub) column (Amersham Biosciences) equilibrated with 50 mM potassium phosphate buffer, pH 7.6 containing 1 M ammonium sulphate. Unbound protein was eluted (as judged by A₂₈₀) and CYP74C3 was eluted at 5 ml/min with a linear gradient (50 ml, 10 min) from 1-0 M ammonium sulphate. Brown fractions (5 ml) with the highest R_{z} were pooled and concentrated to approximately 2 ml using Amicon Ultra 10,000 molecular weight cut-off centrifugal filter devices (Millipore). The concentrate was injected at 0.2. ml/min onto a Hi-load Superdex 75 26/60 gel filtration column equilibrated with 100 mM sodium phosphate buffer, pH 6.5 and eluted with the same buffer at 2 ml/min.

### Substrates and Other Chemicals for CYP74C3

13α-HPOD (13-S-hydroperoxy-9Z,11E,15Z-octadecadienoic acid), 13α-HPOT (13-S-hydroperoxy-9Z,11E,15Z-octadecatrienoic acid), 9α-HPOD (9-S-hydroperoxy-10E,12Z,15Z-octadecadienoic acid) and 9α-HPOT (9-S-hydroperoxy-10E,12Z,15Z-octadecatrienoic acid) were obtained from Larodan (Malmö, Sweden) or from Prof. Mats Hamberg (Karolinska Institute, Stockholm, Sweden). They were stored in sealed vials at a concentration of 5-20 mM in ethanol under argon at −80° C. The exact concentration of substrate was determined using an extinction coefficient at 234 nm of 25000 M⁻¹. cm^{-1 [36}]. Emulphogene, imidazole, benzimidazole (BI), potassium cyanide (KCN), potassium thiocyanate (KCNS), pyridine, dimethylphenylphosphine (DMPhP), thiazole and azide (sodium salt) were purchased from Sigma-Aldrich (Poole, Dorset, U.K.). Carbon monoxide (CO) and O₂ were supplied by BOC gases (Manchester, U.K.). Oligonucleotides were obtained from Invitrogen or Sigma-Genosys (Haverhill, Cambridgeshire, U.K.)

### UV/Visible Spectroscopy

Spectra and steady-state kinetic analyses were performed using a dual beam scanning Shimadzu UV/Visible spectrophotometer (Model UV-1601, Shimadzu, Milton Keynes, U.K.). UV/Visible stopped-flow spectrophotometric experiments were performed using a Hi-Tech SF-61 DX-2 double mixing apparatus (Hi-Tech Scientific, Salisbury, Wiltshire, U.K.) interfaced with a CU-61 control unit installed in an anaerobic glovebox operating under 1 atmos. of N₂ containing 1 ppm O₂. Stopped flow-UV/Visible data were analysed with the KineticAsyst 3.0 software package (Hi-Tech Scientific). Ligand-binding studies were carried out with both the native oxidised and the dithionite ion reduced forms of CYP74C3.

### EPR Spectroscopy

EPR spectroscopy was performed on a Bruker ELEXYS 500 spectrometer with an ER049X SuperX microwave bridge and a shq cavity (Bruker Analytische Messtechnik GmbH). Low temperature experiments were performed using an Oxford Instruments ESR-900 cryostat and ITC3 temperature controller. EPR spectra were simulated using the computer programme SimFonia (Bruker). EPR spin concentration measurements were made by double integration and comparison with a copper EDTA standard under non-saturating conditions. EPR spectra were measured at 10 K and 2 mW microwave power [37].

### Haem Incorporation

Haem content and type was determined from quantification of the pyridine haemochrome using alkali-denatured protein and an extinction coefficient for the reduced-oxidised difference spectrum of 28360 M⁻¹. cm⁻¹ at 556.4 nm [38].

### Standard Activity Assay for CYP74C3

The standard assay mixture (0.5 ml) contained 20 μM substrate in 100 mM sodium phosphate buffer, pH 6.5. The decrease in A₂₃₄ was followed for 20-60 s at 25° C. and converted to moles substrate using an extinction coefficient of 25000 M⁻¹. cm^{-1 [37}]. The initial linear region of the progress curves was used to calculate rates. CYP74C3 concentration was determined from the haem content using a calculated extinction coefficient at 391 nm of 122000 M⁻¹. cm⁻¹. Protein content was estimated using the Bradford [39] or bicinchoninic acid (BCA) assay (Pierce, Perbio Science, Cramlington, Northumberland, U.K.) according to the manufacturer's instructions. Bovine serum albumin was used as a standard.

### Kinetic Properties

Steady state kinetic data were collected using Shimadzu kinetics software (version 2.7). Kₘ and k_{cat} for substrates and K_{d} for Emulphogene were calculated by fitting the data sets to a one site saturation model for simple ligand binding using SigmaPlot 8 (Sigma-Aldrich). Substrate specificity was determined by comparing k_{cat}/Kₘ. Predicted micelle concentration (μM) was calculated using the equation: [Micelle]=([total detergent]−CMC)/N, where aggregation number N=number of monomers/micelle (88 for Emulphogene [40]). The “positive” data was used to estimate the K_{d} for binding of an Emulphogene micelle.

### GC-MS Analysis

The spectrophotometric assay used to measure CYP74C3 activity monitored only substrate disappearance and was unable to distinguish between HPL and AOS activities. Non-volatile products were extracted from reaction mixtures essentially as described [41]. A reaction (10 ml) containing 13α-HPOT (40 μM) and CYP74C3 in detergent-buffer (20 μg), detergent-free CYP74C3 (2 μg) or the product 12-oxo-(9Z)-dodecenoic acid in 100 mM sodium phosphate buffer, pH 6.5 was incubated at 22° C. for 15 min, adjusted to pH 4.3 with dilute acetic acid and applied to a conditioned Sep-Pak C18 cartridge (Waters, Elstree, Hertfordshire, U.K.). Air was then forced through the cartridge to remove water and bound products were eluted with methanol. Reduction, methylation and trimethylsilylation for GC-MS analysis were carried out exactly as described [41]. GC-MS analysis confirmed that detergent-free CYP74C3 was an HPL and not an AOS since 12-oxo-(9Z)-dodecenoic acid was the major non-volatile product from a reaction with 13α-HPOT as substrate (data not shown): m/z (ion attribution; relative intensity), 300 (M+; 0.06%), 285 (M+—CH₃; 3.2%), 253 (M+—CH₃O₂; 11.6%), 103 (CH₂OTMS+; 100%), 73 (TMS+; 91.4%) [41]. The isomerisation product, 12-oxo-(10E)-dodecenoic acid (traumatin) was also detected: m/z (ion attribution; relative intensity), 300 (M+; 1.6%), 285 (M+—CH₃; 8.7%), 253 (M+—CH₃O₂; 51.9%), 129 (C₃H₄OTMS+; 100%) and 73 (TMS+; 81.1%) [41].

Detergent-free CYP74C3 exhibited a broad pH profile with activity maxima between pH 5.5 and 7.5 with all four substrates tested. Activity was still significant outside this range for 13-hydroperoxides, but significantly decreased above pH 8.0 for 9-hydroperoxides. pH 6.5 was selected as the optimum pH for standard assays. For CYP74C3 purified in detergent-buffer, the k_{cat} with 13α-HPOD, 9α-HPOT and 9α-HPOD were 70, 5 and 38% respectively of that calculated with 13α-HPOT (Table 1).

TABLE 1
Kinetic parameters and substrate specificity of
purified CYP74C3 before and after removal of detergent
and after reactivation with detergent

| Kinetic parameter | +Detergent | Detergent-free | Reactivated † |
|---|---|---|---|
| k_{cat} (s⁻¹) * | | | |
| 13α-HPOT | 514 ± 11 | 68 ± 5 | 657 ± 14 |
| 13α-HPOD | 361 ± 19 | 59 ± 3 | 493 ± 17 |
| 9α-HPOT | 24 ± 2 | 8 ± 0 | 30 ± 1 |
| 9α-HPOD | 197 ± 6 | 44 ± 2 | 185 ± 7 |
| Kₘ (μM) | | | |
| 13α-HPOT | 3.3 ± 0.4 | 20.9 ± 1.7 | 4.4 ± 0.6 |
| 13α-HPOD | 10.3 ± 2.1 | 102.0 ± 11.8 | 14.7 ± 1.9 |
| 9α-HPOT | 39.1 ± 7.2 | 81.6 ± 9.7 | 51.7 ± 4.4 ‡ |
| 9α-HPOD | 15.3 ± 1.4 | 84.5 ± 10.1 | 25.8 ± 2.2 ‡ |
| k_{cat}/Kₘ (μM⁻¹ · s⁻¹) | | | |
| 13α-HPOT | 155.8 | 3.2 | 149.3 |
| 13α-HPOD | 35.0 | 0.6 | 33.5 |
| 9α-HPOT | 0.6 | 0.1 | 0.6 |
| 9α-HPOD | 12.9 | 0.5 | 7.2 |
| Substrate specificity | | | |
| (% 13α-HPOT) | | | |
| 13α-HPOT | 100 | 100 | 100 |
| 13α-HPOD | 23 | 19 | 22 |
| 9α-HPOT | 0.4 | 3 | 0.4 |
| 9α-HPOD | 8 | 16 | 5 |

* Assuming 1 active site per monomer of Mᵣ 56.8 kDa
† With 5 mM Emulphogene
All data were fitted for substrate concentrations up to 160 μM, except ‡ where tighter fits were achieved using the data for concentrations up to 80 μM.
Error bars are the means ± standard errors of at least six determinations on two different enzyme preparations

A comparison of the catalytic efficiencies (k_{cat}/Kₘ) of CYP74C3 with the four substrates (Table 1) confirms, however, that the preferred substrate by far was 13α-HPOT, and the efficiency of turnover of 13α-HPOD, 9α-HPOT and 9α-HPOD were only 23, 0.4 and 8% respectively of that with 13α-HPOT. With 13α-HPOT as the substrate, the k_{cat} of CYP74C3 in detergent-buffer was approximately 8-fold higher than detergent-free enzyme (Table 1) and was associated with a 6-fold decrease in affinity for this substrate. The effects of detergent removal on the activity of CYP74C3 with the other three substrates were similar. For 13α-HPOD, k_{cat} and Kₘ were 6- and 10-fold lower and higher respectively; for 9α-HPOT, k_{cat} and Kₘ were 3- and 2-fold lower and higher respectively and for 9α-HPOD the k_{cat} and Kₘ were 5- and 6-fold lower and higher respectively. Detergent removal reduced the catalytic efficiency of CYP74C3, as calculated from k_{cat}/Kₘ, to a level that was only 2% (13α-HPOT and 13α-HPOD), 17% (9α-HPOT) and 4% (9α-HPOD) of that observed when the enzyme was purified without detergent removal; there was no major effect of detergent removal on substrate specificity: 13α-HPOD (19%, detergent present; 23% detergent-free), 9α-HPOT (0.4%, detergent present; 3% detergent-free) and 9α-HPOD (8%, detergent present; 16% detergent-free).

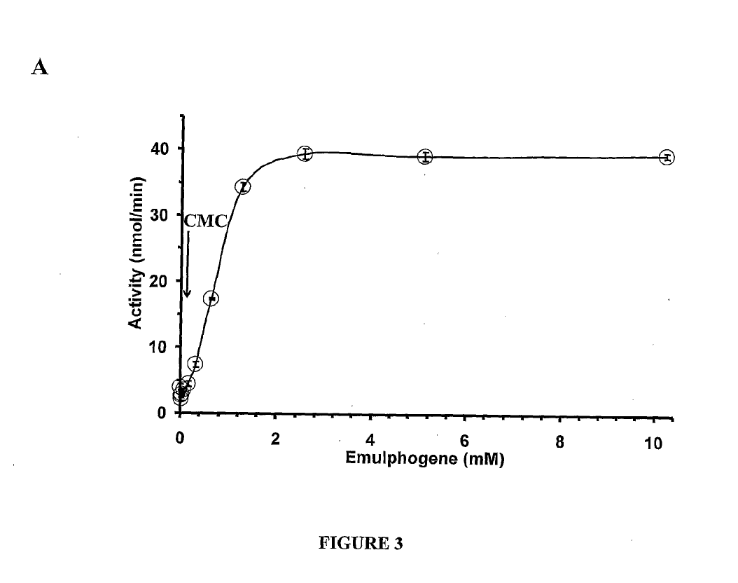

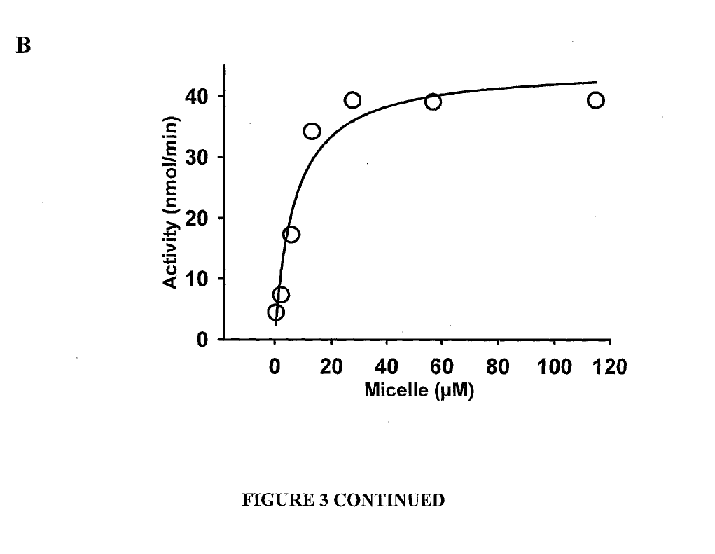

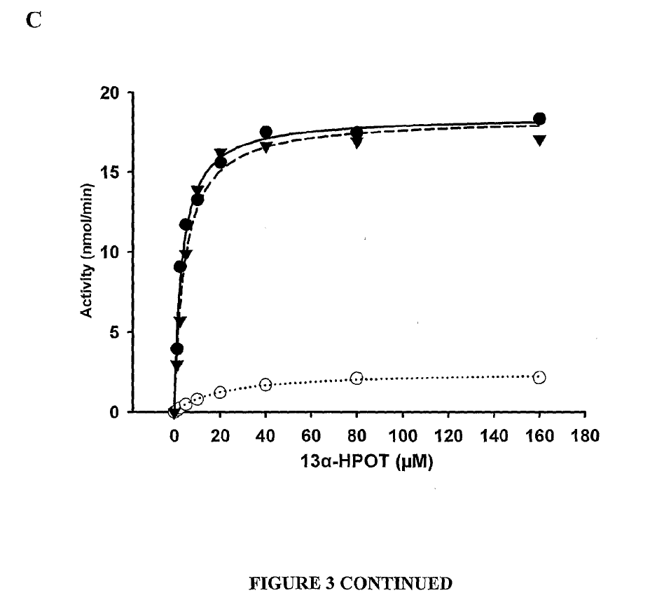

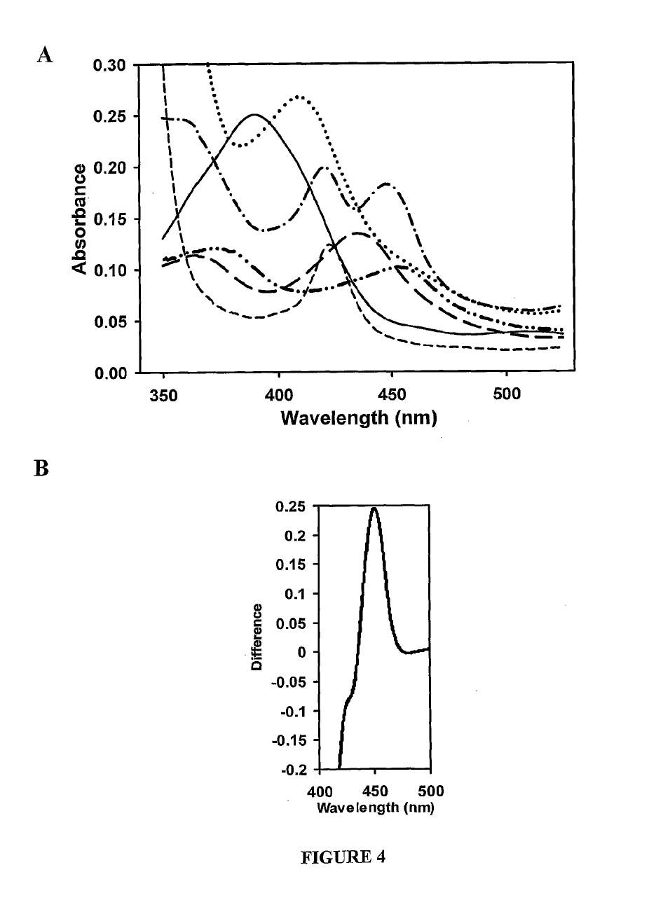

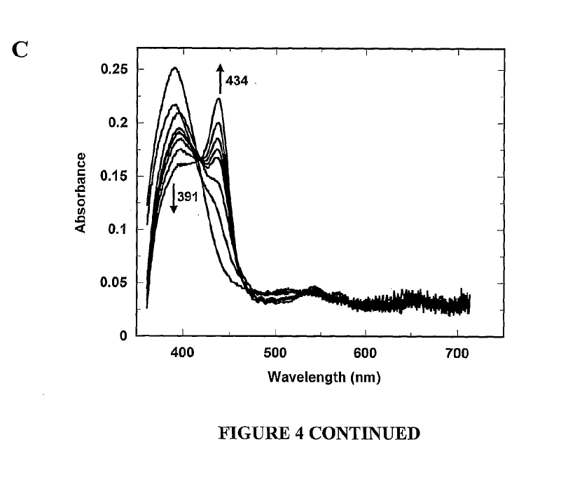

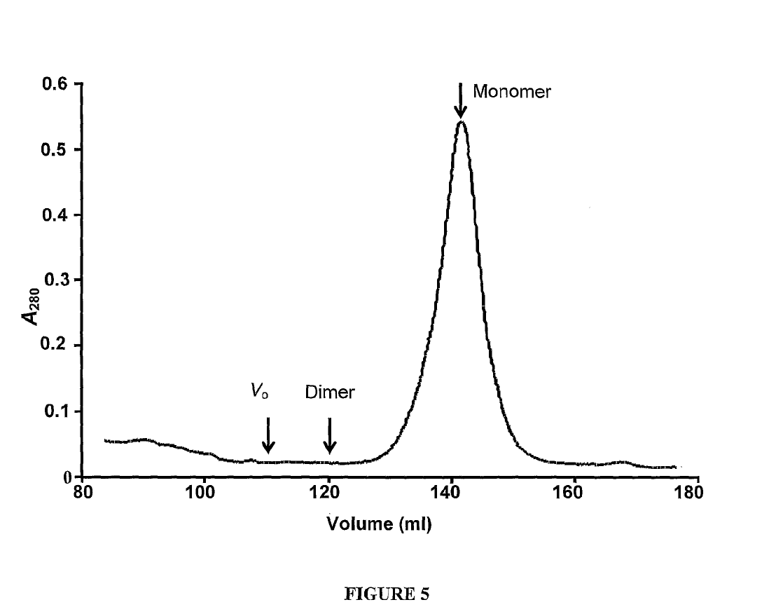

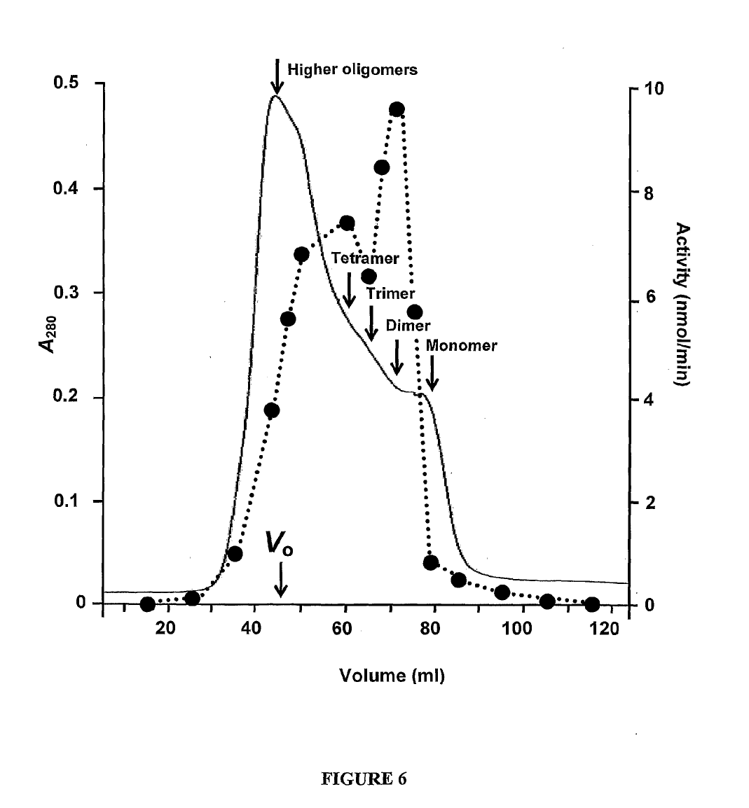

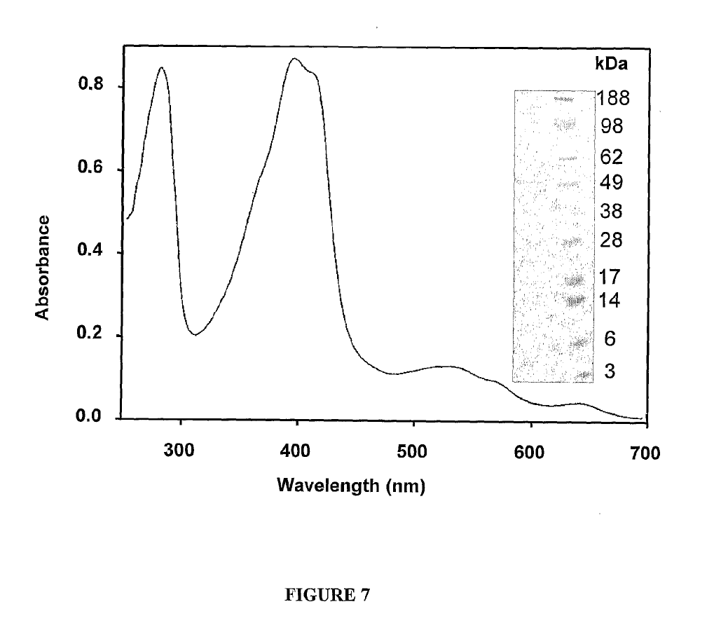

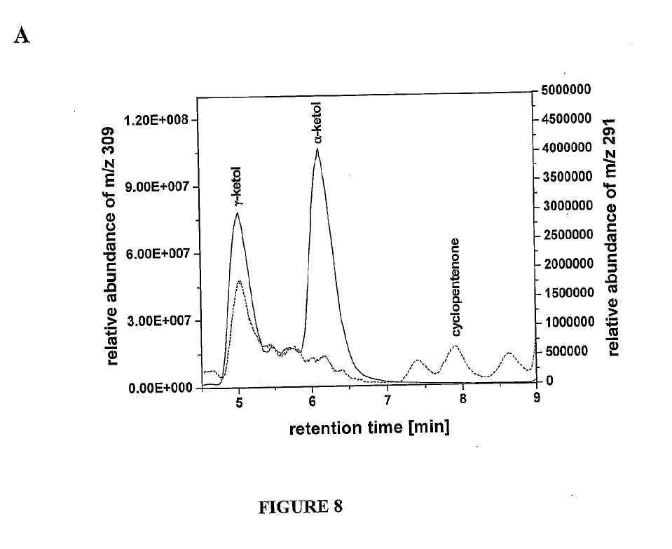

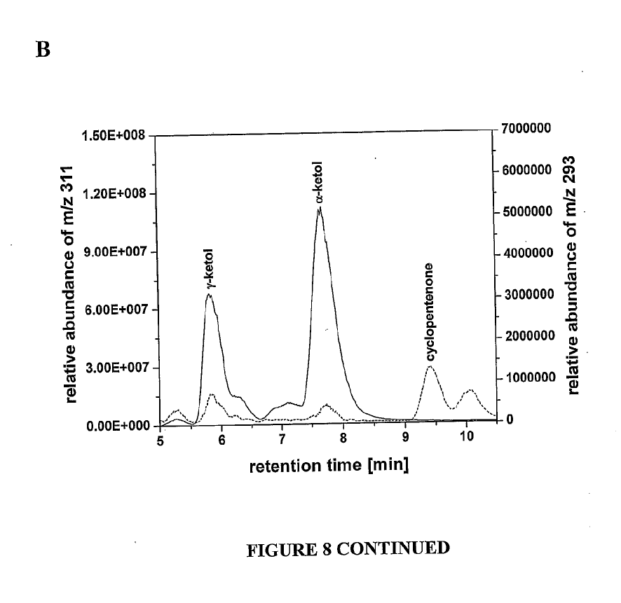

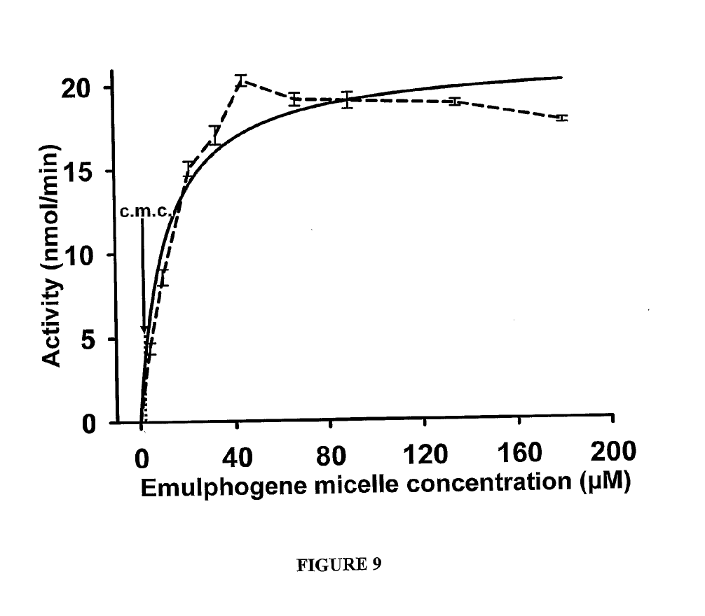

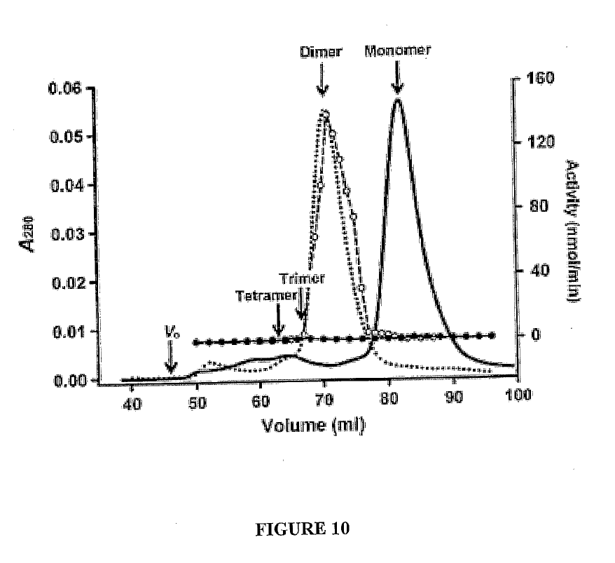

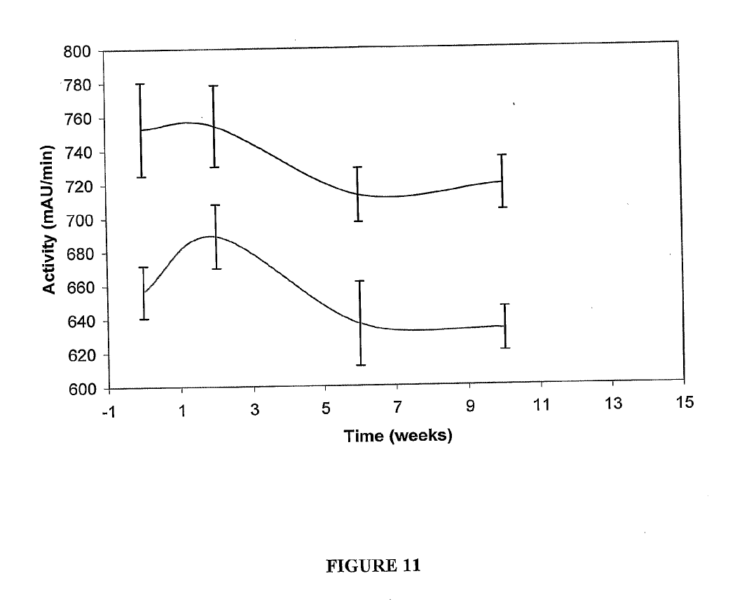

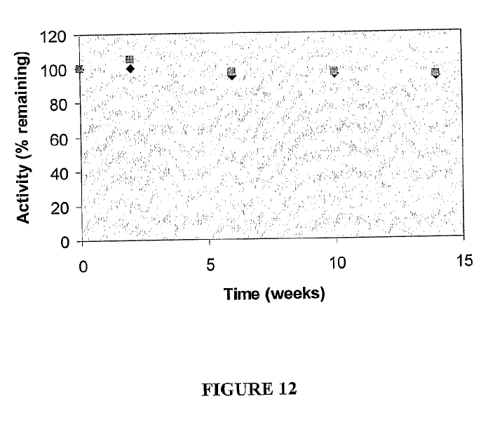

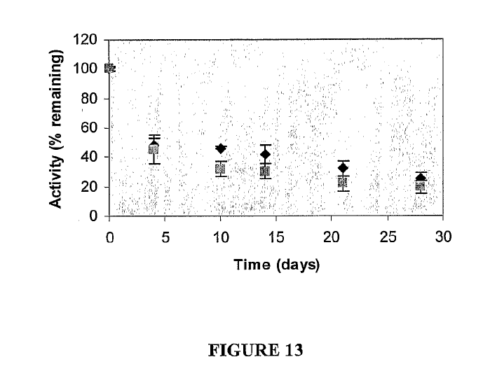

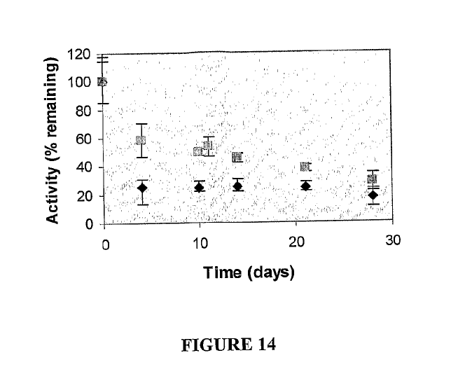

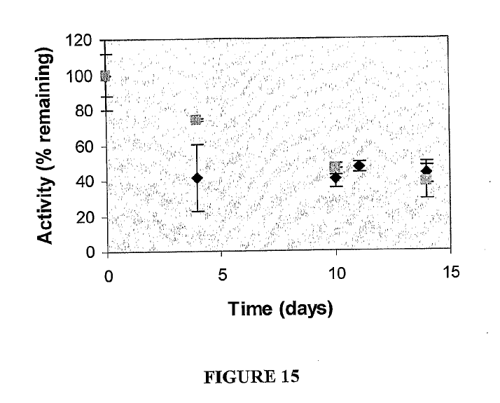

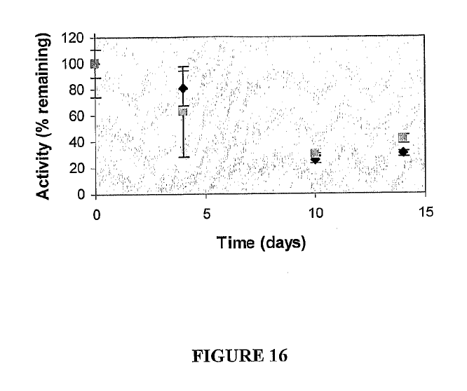

Detergent-free CYP74C3 could be reactivated with the detergent Emulphogene at a concentration exceeding 2.6 mM (FIG. 3a). No reactivation was observed with Emulphogene at a concentration of 0.1 mM, which was slightly below the CMC. The K_{d} for the binding of Emulphogene micelles to CYP74C3 was estimated as 6.9±1.1 λM (FIG. 3b). The k_{cat}, Kₘ and k_{cat}/Kₘ values for reactivated CYP74C3 with all four substrates were very similar to those recorded for CYP74C3 purified without detergent removal (Table 1), so, as observed for detergent removal, reactivation with detergent also had no effect on substrate specificity. Reactivation of detergent-free CYP74C3 was observed with concentrations of 13α-HPOT (and the other three substrates, data not shown) up to 160 μM (FIG. 3c); at concentrations >240 μM there was substantial substrate inhibition (data not shown). These substrates may, however, start to form micelles at concentrations well above this concentration, though no CMC, or behaviour to form micelles, has been reported. Sephadex G-50 chromatography indicated that aqueous solutions of 13α-HPOT at concentrations up to 80 mM, Vitamin B12 and 2,4-dinitrophenyl-DL-methionine sulfoxide were all retained to some degree by a Sephadex G-50 column and eluted at 2.16-2.32 ml; in contrast, blue dextran, carbonic anhydrase, detergent-free CYP74C3 monomer and Emulphogene micelle, were all excluded from the matrix and eluted at 1.00-1.09 ml. A sample containing equimolar amounts (5 mM) of 13α-HPOT and Emulphogene separated as two overlapping peaks with elution volumes of 1.12 ml and 2.09 ml, very similar to that of the Emulphogene micelle and 13α-HPOT monomer respectively (data not shown). These data showed that 13α-HPOT did not form any micelles, or mixed micelles with Emulphogene.

The effects of ligand binding or reduction by sodium dithionite on the UV/Visible spectrum and the spin state of the haem iron on detergent-free CYP74C3 are summarised in Table 2.

TABLE 2
Effects of reduction and ligand- or detergent-binding on the UV/Visible spectrum and spin
state of the haem iron for detergent-free CYP74C3 and a comparison with other CYP74
enzymes, CYP2B4 and CYP1A2

| | | Ligand | Soret | Visible | Spin | |
|---|---|---|---|---|---|---|
| Cytochrome | Ligand or Detergent | (mM) | (nm) | (nm) | state | Reference |
| CYP74C3 | Native | — | 391 | 508, 545 | High | This work |
| | Reduced | — | 408 | 547 | High | This work |
| | Emulphogene (5.0 mM) | — | 393, 420(s) | 568, 545(s) | Low | This work |
| | Imidazole | 480 | 366, 426 | 544, 547 | Low | This work |
| | KCN | 500 | 365, 434 | 539, 566 | Low | This work |
| | Thiazole | 500 | 422 | 537, 564 | Low | This work |
| | Reduced + CO | ~1 | 452 | 560 | Low | This work |
| | O₂ | ~1 | 422 | 535, 564 | Low | This work |
| | DMPhP | 250 | 375, 453 | 563 | Low | This work |
| | Pyridine | 375 | 422 | 480, 557 | Low | This work |
| | BI | 250 | 423 | 541, 576 | Low | This work |
| | KCNS | 1000 | 374, 414 | 530 | Mixture | This work |
| | Azide | 1000 | 407 | 553 | Mixture | This work |
| CYP74B1 | Native | — | n.d. | n.d. | n.d. | [24] |
| (Pepper | Triton (3.2 mM) | — | 393 | 512, 540 | High | [24] |
| HPL) | Reduced + Triton | — | 410 | 550 | High | [24] |
| | Imidazole + Triton | 540 | 360, 424 | 544, 576 | Low | [24] |
| | KCNS + Triton | 200 | 364, 433 | 555 | Low | [24] |
| | Reduced + Triton + NO | ~1 | 431 | 541, 571 | Low | [24] |
| CYP74B3 | Native | — | n.d. | n.d. | n.d. | [21] |
| (Tomato | Triton (4.8 mM) | — | 390 | 500(s) | Low | [21] |
| HPL) | Reduced + Triton | — | 405 | 550(s) | Low | [21] |
| | Reduced + Triton + CO | ~1 | 447 | No feature | Low | [21] |
| CYP74B4 | Native | — | 418 | No feature | Low | [13, 49] |
| (Alfalfa | Reduced | — | 420 | No feature | Low | [13, 49] |
| HPL) | Triton (3.2 mM) | — | 390 | No feature | High/low* | [49] |
| | Reduced + Triton | — | 416 | No feature | Low | [49] |
| | Pyridine | — | 390 | No feature | Low | [49] |
| | Pyridine + Triton | — | 418 | No feature | Low | [49] |
| CYP74C2 | Native | — | n.d. | n.d. | n.d. | [28] |
| (Melon | Emulphogene (1.6 mM) | — | 418 | 537, 570 | Low | [28] |
| HPL) | Reduced + Emulphogene + | ~1 | 447 | No feature | Low | [28] |
| | CO | | | | | |
| Flax AOS† | Native | — | n.d. | n.d. | n.d. | [48] |
| | Emulphogene (1.6-4.0 mM) | — | 391 | 512, 540 | High | [48] |
| | Reduced + Emulphogene | — | 407 | No feature | High | [48] |
| | Reduced + Emulphogene + | ~1 | 450 | No feature | Low | [48] |
| | CO | | | | | |
| AOS | Native | — | 406 | 500, 534 | High | [47] |
| domain of | Reduced + CO | ~1 | 427 | 544, 569 | Low | [47] |
| coral AOS- | Azide | 200 | 408 | 500 | Mixture | [47] |
| LOX fusion | KCNS | 200 | 370, 420 | 550, 578 | Low | [47] |
| (Coral | | | | | | |
| AOS) | | | | | | |
| CYP2B4 | Native | — | 418 | 535, 568 | Low | [50, 53] |
| | Reduced | — | 413 | 544 | Low | [50] |
| | n-octyl glucoside (10 mM) | — | 425 | No feature | Low | [54] |
| | Imidazole | 50 | 359, 426 | No feature | Low | [50] |
| | BI | 0.05 | 359, 423 | 540, 574 | Low | [50] |
| | DMPhP | 0.5 | 377, 452 | 560 | Low | [50] |
| | KCN | 50 | 365, 436 | 559 | Low | [50] |
| | Reduced + CO | ~ 1 | 368, 451 | 554 | Low | [50] |
| CYP1A2 | Native | — | 394 | No feature | High | [50, 53] |
| | Reduced | — | 411 | 542 | Low | [50] |
| | Renex 690 (1.6-8.1 mM) | — | 418 | No feature | Low | [53] |
| | Imidazole | 200 | 356, 424 | No feature | Low | [50] |
| | BI | 1.3 | 361, 423 | 539, 575 | Low | [50] |
| | DMPhP | 0.33 | 370, 450 | ~550 | Low | [50] |
| | KCN | 90 | 368, 436 | 553 | Low | [50] |
| | Reduced + CO | ~1 | 366, 447 | 549 | Low | [50] |

CYP74C3 concentration was 3.25 μM, except for reactions of CYP74C3 with Emulphogene, which were at 10.2 μM and all reactions with CYP74C3 were carried out in 100 mM sodium phosphate buffer, pH 6.5 at 25° C.;
(s), shoulder;
*Discrepancy between EPR and UV/Visible spectroscopy data;
†CYP74A1 was not characterised as a recombinant enzyme;
n.d., not determined.

For selected ligands, the UV/Visible spectra are shown in FIG. 4a. A shift in the UV/Visible spectrum to 452 nm (FIG. 4b) was observed after binding CO to the dithionite-reduced enzyme, which is typical of P450-like enzymes. The dithionite-reduced enzyme exhibited a shift in the Soret band from 390 to 408 nm, consistent with high spin Fell. The UV/Visible spectra of CYP74C3 with cyanide or imidazole bound were typical of a low spin haem iron with red shifts for both Soret and visible bands; the cyanide complex (FIG. 4c) exhibited bands at 365, 434, 539 and 566 nm and the imidazole complex exhibited bands at 366, 426, 544 and 547 nm (data not shown). The time course for cyanide binding monitored at 440 nm using the stopped-flow diode array data shown in FIG. 4c was biphasic with fits to two exponential functions giving k₁=7.9±0.3×10⁻² s⁻¹ (amplitude 0.045) and k₂=1.3±0.1×10⁻² s⁻¹ (amplitude 0.13). The spectra associated with the second phase exhibited an isosbestic point at 419 nm. The kinetics of imidazole binding were also biphasic (data not shown) with k₁=2.5±0.1×10⁻² s⁻¹ (amplitude 0.04) and k₂=0.33±0.03×10⁻² s⁻¹ (amplitude 0.024). Ferrous low spin haem iron spectrophotometric signatures were also observed for CO, O₂, pyridine and BI with red shifted Soret and visible bands (Table 2). Binding of the sulphur- and phosphorous-containing ligands thiazole and DMPhP gave low spin haem iron bands with large Soret band shifts (Table 2); the DMPhP complex has bands at 375, 453 and 563 nm (FIG. 4a). Thiocyanate and azide binding resulted in a mixture of high and low spin haem iron bands (Table 2).

EPR spectroscopy indicated that the resting enzyme comprised a mixture of high and low spin haem iron spectra with g factors of 8.03, 3.51, 1.68 and 2.39, 2.24, 1.93 respectively (values from simulation of spectra) in the ratio of 2.5:1. The addition of 13α-HPOT resulted in a 60% loss of EPR intensity and a ratio of high and low spin haem iron of 1:2. Addition of Emulphogene at a concentration of 2.4 mM, well above the CMC, to resting enzyme (240 μM), resulted in a change in the ratio of high and low spin haem iron to 1:5.5, but with no change in the total EPR concentration. Subsequent addition of 13α-HPOT at the same concentration resulted in a 50% reduction in the total concentration and a ratio of high spin to low spin haem iron signal of 1:6. Similar effects were also seen with resting enzyme at 24 μM, which suggests that there was no concentration dependency. At a concentration of 0.1 mM, slightly below the CMC of Emulphogene, both Emulphogene and 13α-HPOT had no effect on the EPR spectrum of resting enzyme at 24 μM. Changes in the spin state of the haem iron, induced by detergent micelles, were also associated with subtle changes in the UV/Visible spectrum. Addition of 1.56 mM Emulphogene to the detergent-free enzyme at 10.2 μM resulted in a change in the Soret region; the peak at 391 nm was shifted to 393 nm and a shoulder developed at 420 nm with an isosbestic point at 401 nm. Similar changes at longer wavelengths were observed; the features at 508 nm and 644 nm were reduced, the feature at 545 nm increased slightly and a new feature appeared at 568 nm with isosbestic points at 454, 530, 581 and 669 nm. No clear changes in the UV/Visible spectrum were observed during the reaction of 10.2 μM CYP74C3 in the presence and absence of 5 mM Emulphogene, with 100 μM or 2.4 mM 13α-HPOT, nor with detergent-free 10.2 μM CYP74C3 and 0.08 mM Emulphogene.

### Protein Sequencing and SDS-PAGE Analysis

The amino acid sequence of CYP74C3 predicted from the cDNA sequence was confirmed by a combination of MALDI-ToF, Edman sequencing and In Source Decay (ISD) (E. Belfield, R. Hughes, I. Galetich, K. Wilson, M. Naldrett and R. Casey, unpublished work). SDS-PAGE gel electrophoresis was carried out using NuPAGE (4-12% w/v gradient Bis-Tris gels) with MES SDS running buffer and SeeBlue Plus 2 protein markers according to the manufacturer's instructions (Invitrogen).

### Gel Filtration

The relative molecular mass (Mᵣ) (relative to protein standards) of native CYP74C3 in detergent-free buffer was determined by gel filtration on a Superdex 75 26/60 column. The Mᵣ of the unknown was determined from a plot of log Mᵣ (y) versus Vₑ/Vₒ (x) and the data fitted by linear regression (correlation coefficient 0.996) to the equation: y=−1.2496×+6.4101. The Mᵣ of native CYP74C3 in detergent-buffer was determined on a Superdex 200 16/60 column. The data were fitted by linear regression (correlation coefficient 0.988) to the equation: y=−1.5385x+7.4166.

Gel-filtration of CYP74C3 in 100 mM sodium phosphate buffer, pH 6.5 containing Emulphogene at 0.08 mM, slightly below the critical micelle concentration (CMC) of 0.125 mM [40] was also carried out using a calibrated Superdex 200 HR 10/300 column (Amersham Biosciences) eluted at 0.5 ml/min.

### Analytical Ultracentrifugation

Sedimentation velocity and equilibrium experiments were carried out to determine the effects of protein concentration, ionic strength, pH and detergent-buffer on the oligomeric state of CYP74C3.

Sedimentation velocity analysis of detergent-free CYP74C3 (0.5 and 1.0 mg/ml in 100 mM sodium phosphate buffer, pH 6.5) was carried out in a Optima XL-A ultracentrifuge (Beckman Coulter, High Wycombe, Buckinghamshire, U.K.) at 50,000 rpm and 20.0° C., with solute distributions recorded using UV-absorption optics at 280 nm with scans every 3.5 min. Experiments were repeated using CYP74C3 at the same protein concentrations and also from 5 to 15 mg/ml using the interference optical system in an XL-I ultracentrifuge (Beckman Coulter). Similar experiments on CYP74C3 (1 mg/ml) were carried out as above after buffer exchange into 10 mM, 50 mM or 100 mM sodium phosphate buffer, pH 6.5 and into 100 mM sodium phosphate buffer at pH 5.8, 6.9 and 7.8. To study the effects of detergent-buffer on CYP74C3 oligomerisation, sedimentation velocity experiments were carried out as above with CYP74C3 (0.5 and 1 mg/ml) in detergent-buffer. Scans were recorded at 232 nm every 3.5 min. The raw sedimentation data were analysed using the procedure SEDFIT [42]. Both the c(s) and least square g(s) models were employed for the analysis of the sedimentation data and for the determination of the apparent sedimentation coefficient. Because of the very heterogeneous nature of CYP74C3 in association with detergent, an overlay of the c(s) model on the least square g(s) distribution was used to identify the peaks in the least square g(s) plot. In addition, multiple Gaussians were fitted to the least square g(s) profile obtained from SEDFIT via MULTIG using the “Robust” fitting algorithm to allow an approximation of the proportion of the oligomers present in solution. The Optima XL-A ultracentrifuge (Beckman Coulter) was also used to determine the weight average molecular weight, M_{w}, of detergent-free CYP74C3 using low speed sedimentation equilibrium. Samples (0.5 or 1 mg/ml detergent-free CYP74C3 in 100 mM sodium phosphate buffer, pH 6.5) were centrifuged at 20,000 rpm and 20.0° C. and scans were taken at 280 nm every 2 h until equilibrium was reached (after 24 h). To obtain the M_{w} of CYP74C3 in detergent-buffer, data were analysed using the MSTARA algorithm [43]. The effects of thermodynamic non-ideality were assumed to be negligible.

### Other Parametric Calculations

The partial specific volume ( ν̅) of detergent-free CYP74C3 in 100 mM sodium phosphate buffer, pH 6.5 was calculated from the predicted amino acid composition using the routine SEDNTERP [44] to be 0.744 ml/g. The partial specific volume of HPL-F in detergent-buffer was measured by determination of the relative viscosity and density using a viscometer and an Anton-Paar density meter respectively as this included a contribution from micellar association of the protein. The average Mᵣ of an Emulphogene micelle has been reported to be 56.0 kDa [40]. We also made our own approximation by applying the empirical equations of Squire and Himmel [45] in the software BIOMOLS (http://www.nottingham.ac.uk/ncmh/unit/method.html#Software). On the assumption that micelles are globular particles with a sedimentation coefficient close to 1S and using the calculated value for ν̅ of 0.945 ml/g, the Mᵣ of an Emulphogene micelle was ˜68 kDa. An average value of ˜62 kDa was used for further calculations

### Gel-Filtration Analysis of Micelle Formation

An aqueous solution of 13α-HPOT in 100 mM sodium phosphate buffer, pH 6.5 (80 mM, 30 μl) with and without an equimolar amount of Emulphogene (80 mM) was injected onto a Sephadex G-50 (Amersham Biosciences) column (27×0.275 cm, 1.6 ml) in 100 mM sodium phosphate buffer, pH 6.5 and eluted at 0.1 ml/min. Standards (30 μl) of known Mᵣ were: Emulphogene detergent micelle (62 kDa, 80 mM), detergent-free CYP74C3 monomer (56.8 kDa, 20 μM); carbonic anhydrase (29 kDa, 3 mg/ml); Vitamin B12 (1.355 kDa, 0.1 mg/ml), blue dextran (2000 kDa, 2 mg/ml) and N-2,4-dinitrophenyl-DL-methionine sulfoxide (331.3 Da, 0.1 mg/ml).

### Oligomeric State of Detergent-Free CYP74C3

The Mᵣ of histidine-tagged CYP74C3, based on the protein sequence and predicted from the cDNA, was 56.8 kDa. This was consistent with the value determined for the native protein by gel filtration (relative to globular protein standards) of 55.2 kDa (FIG. 5). These measurements, and dynamic light scattering (data not shown), indicated that detergent-free CYP74C3 was a monomer. No dimer, which, if present, would have been cleanly separated from monomer, could be detected by gel filtration (FIG. 5) and activity could be detected only in the monomer fraction. Under the conditions in the ultracentrifuge, analysis of the sedimentation velocity data indicated that detergent-free CYP74C3 at 1 mg/ml was also almost exclusively a monomer with a sedimentation coefficient of 3.5S (data not shown). A corresponding weight average molecular weight, M_{w}, of (55±2) kDa was determined from sedimentation equilibrium studies on CYP74C3 at 1 mg/ml, in excellent agreement with the gel filtration result. At higher protein concentrations (up to 15 mg/ml) a small amount of aggregated protein (5 to 11S) was detected by sedimentation velocity. In addition, there was some evidence for the presence of dimers, but these were not in reversible equilibrium with the monomers (increase in loading concentration caused no increase of dimers relative to monomers). CYP74C3 at 15 mg/ml was therefore almost exclusively monomeric with an average sedimentation coefficient of 3.7S. As the ionic strength was decreased from 100 mM to 10 mM the proportion of monomer in the sample decreased and the proportion of aggregated protein (5 to 11S) generally increased. The proportion of monomer also increased with an increase in pH from 5.8 to 7.9. Again, some aggregated protein (5 to 10S) was present along with the monomer.

We carried out a gel filtration analysis of untagged CYP74C3, which confirmed the presence of active monomers, plus a small amount of soluble aggregated protein that eluted at the void volume (data not shown), exactly as observed for the tagged protein. This suggested that the histidine-tag plus Gateway® att recombination sequence had no effect on the oligomeric state. Similarly, CYP74C3 expressed without the first 11 amino acids at the N-terminus (MASSSETSSTN) was also almost exclusively monomeric (data not shown), suggesting that the N-terminal extension sequence of CYP74C3 was not involved in determining oligomeric state.

### Oligomeric State of CYP74C3 in Detergent-Buffer

Gel filtration analysis of CYP74C3 in detergent-buffer indicated that it formed only a small amount of monomer, as judged from the peak position on a calibrated Superdex 200 16/60 column (FIG. 6). The smallest active oligomer eluted at a position corresponding to the size of a protein dimer, with most of the protein forming a heterogeneous mixture of non-resolved higher oligomers. In order to attempt to resolve the sizes of the individual components in the mixture, the sample was analysed by analytical ultracentrifugation, which confirmed the gel filtration analysis and indicated that CYP74C3 formed a broad range of oligomers . The peaks identified in the g(s) distribution using the sedimentation velocity data for CYP74C3 at 0.5 mg/ml indicated that there were several oligomers present with apparent sedimentation coefficients of 3.2, 5.3, 7.0, 9.4, 12.1, 14.8 and 17S. These were assigned only where peaks in the c(s) distribution coincided with peaks in the g(s) distribution. It is plausible that the formation of micelle-protein complexes would allow sedimentation to occur more slowly than the pure protein because of the high partial specific volume of the detergent. We may note that on the basis of the 3.2S species being monomeric, then on the basis of the frictional ratio being constant over all species a ‘ladder’ of incremental n-mers would have predicted sedimentation coefficients of 3.2, 5.0, 6.5, 8.1, 9.4, 10.6 & 11.7S. The rather higher values seen probably reflect a growing protein:detergent ratio in the micelles as the order of oligomerisation increases. It is very possible that this happens via successive monomers being added into existing micellar units. A comparison of the activities of CYP74C3 oligomers after gel filtration in buffer containing Emulphogene at 1.56 mM, well above the CMC, indicated that most activity was found at a peak position corresponding to the size of a dimer (FIG. 6). Considerable activity was also associated with enzyme eluting at peak positions equivalent to the size of the trimer and tetramer. Lower activity was associated with the soluble aggregates eluting close to the void volume; the activity at the peak position corresponding to the size of the monomer was very low. Specific activities at the peak position of each oligomer were calculated from estimating enzyme content by extinction at 391 nm. This indicated that the oligomer with the highest specific activity eluted at a peak position corresponding to the size of a dimer; the trimer, tetramer, higher oligomers and monomer had 67, 51, 20 and 10% respectively of the activity of the dimer.

When CYP74C3 in detergent-buffer was applied to a gel filtration column equilibrated under detergent-free conditions, a number of higher oligomers were detected, but the smallest, most active, species eluted at a position corresponding to a relative Mᵣ of ˜87 kDa. Gel-filtration analysis of CYP74C3 in 100 mM sodium phosphate buffer, pH 6.5 containing Emulphogene at 0.08 mM (slightly below the CMC) confirmed that it was almost entirely monomeric as observed for the detergent-free protein (data not shown). The specific activity and kinetic parameters of detergent-free CYP74C3 expressed with and without the first 11 amino acids were indistinguishable from those for the full-length protein.

### Example 2

### Characterization of CYP74A1

### Cloning and Expression of CYP74A1

A full-length CYP74A1 cDNA clone (U17068) was obtained from the Arabidopsis Biological Resource Centre (ABRC), Ohio State University, Columbus, Ohio 43210, U.S.A. and cloned into the destination vector pDEST14 using Gateway® technology (Invitrogen) according to the manufacturer's instructions to give the plasmid pDEST14AOS5. The expressed protein had a 4×His-tag at the C-terminus of the protein but lacked the sequence encoding a 32 N-terminal amino acid sequence (MASISTPFPISLHPKTVRSKPLKFRVLTRPIK), which was predicted by ChloroP [16′] to be a chloroplast targeting sequence; this sequence was replaced with just an initiating ATG codon. The protein sequence encoded by the Gateway® attB1 recombination sequence was not incorporated into the expressed protein.

### Extraction and Purification of CYP74A1

Cultures (20×2 L conical flasks each containing 1 L LB-G and 50 μg/ml ampicillin) of E. coli strain BL21 (DE3) transformed with expression plasmid were grown and induced as described for CYP74C3 [15′]. Detergent-free CYP74A1 was extracted and purified using immobilised metal affinity chromatography and gel filtration as described for CYP74C3 [15′]. The RZ of the final preparation (detergent-free) was 1.1. Detergent-free enzyme in 100 mM sodium phosphate buffer, pH 6.5 was concentrated using Amicon Ultra 10 kDa molecular weight cut-off centrifugal filter devices (Millipore) to approximately 100 μM, snap frozen in liquid nitrogen in 100 μl aliquots and stored at −80° C.

### Substrates and Other Chemicals for CYP74A1

b 13-HPOTE (13-S-hydroperoxyoctadeca-9Z, 11 E,15Z-trienoic acid) and 13-HPODE (13-S-hydroperoxyoctadeca-9Z, 11 E-dienoic acid) were purchased from Larodan or provided by Prof. Mats Hamberg (Karolinska Institute, Stockholm, Sweden). They were stored in sealed vials at 20 mM in ethanol under argon at −80° C. The exact concentration of substrate was determined-using an extinction coefficient at 234 nm of 25000 M⁻¹.cm^{-1 [17}′]. Emulphogene was purchased from Sigma-Aldrich. Oligonucleotides were obtained from Invitrogen or Sigma-Genosys.

### UV/Visible and EPR Spectroscopy for CYP74A1

UV/Visible spectra and steady-state kinetic analyses were performed using a dual beam scanning UV/Visible spectrophotometer (Model UV-1601, Shimadzu). EPR spectroscopy was performed on a Bruker ELEXSYS 500 spectrometer with an ER049X SuperX microwave bridge and a shq cavity (Bruker). Low temperature experiments were performed using an Oxford Instruments ESR-900 cryostat and ITC3 temperature controller. EPR spectra were simulated using the computer programme SimFonia (Bruker). EPR spin concentration measurements were made by double integration and comparison with a copper EDTA standard under non-saturating conditions [18′]. EPR spectra were measured at 10 K and 2 mW microwave power.

### Standard Activity Assay for CYP74A1

The standard assay mixture (0.5 ml) contained 20 μM substrate in 100 mM sodium phosphate buffer, pH 6.5. The decrease in A₂₃₄ was followed for 20-60 s at 25° C. and converted to moles substrate using an extinction coefficient of 25000 M⁻¹.cm^{-1 [}17′]. The initial linear region of the progress curves were used to calculate rates. CYP74A1 concentration was determined from the haem content [19′] using a calculated extinction coefficient at 391 nm for the detergent-free protein of 89000±7000 M⁻¹.cm⁻¹. Protein content was estimated using the Bradford assay [20] with bovine serum albumin as a standard.

### Kinetic Properties of CYP74A1

Steady state kinetic data were collected using Shimadzu kinetics software (version 2.7). Kₘ and k_{cat} for substrates and K_{d} for Emulphogene were calculated by fitting the data sets to a one site saturation model for simple ligand binding using SigmaPlot 8 (Sigma-Aldrich). K_{d} measurements were carried out in 100 mM sodium phosphate buffer, pH 6.5. Predicted micelle concentration (μM) was calculated using the equation: [Micelle]=([total detergent]-c.m.c.)/N, where aggregation number N=number of monomers/micelle (88 for Emulphogene [21′]). The c.m.c. of Emulphogene has been calculated as 0.125 mM (total detergent concentration) [21′] equivalent to an actual micelle concentration of 1.25 μM. The “positive” data were used to estimate the K_{d} for binding of an Emulphogene micelle. The concentration of Emulphogene described in the text refers to the actual micelle concentration and not the total detergent concentration.

### Gel Filtration of CYP74A1

The molecular mass of native CYP74A1 in 100 mM sodium phosphate buffer, pH 6.5, and in detergent-buffer containing 15.6 μM Emulphogene [15′], was determined by gel filtration on a calibrated Superdex 200 16/60 column. The molecular mass of unknowns was determined from a plot of log molecular mass (y) versus Vₑ/Vₒ (x) and the data were fitted by linear regression (correlation coefficient 0.988) to the equation: y=−1.5385x+7.4166.

### Product Specificity —CYP74A1

For the analysis of AOS products, CYP74A1 (40 μmol), before and after reactivation with 50 μM Emulphogene micelle, was incubated with 13-HPODE, 13-HPOTE, 9-HPODE or 9-HPOTE in 0.1 M sodium phosphate buffer, pH 6.5 for 30 min at 37° C. (and stopped by acidification to pH 3 with acetic acid). The substrate concentrations chosen were 10-fold higher than the Kₘ according to Table 1′. Product analysis was performed by a reversed-phase HPLC/MS Surveyor HPLC system equipped with a photo diode array detector coupled to a LCQ Advantage electrospray ionization ion trap mass spectrometer (Thermo Finnigan, San Jose, Calif., U.S.A.). AOS products were separated by a EC 250/2 Nucleodure 100-5 C18ec column (250×2.1 mm, 5 μm particle size) (Macherey and Nagel, Düren, Germany) with a methanol:water:acetic acid (85:15:0.1, v/v/v) solvent system at a flow rate of 0.18 ml/min. For detection, the mass spectrometer was operated in negative mode with the source voltage set to 4 kV, and a capillary voltage of 27 V and 300° C. In full MS mode, scans were collected between m/z values of 50 and 350. In parallel, the methyl esters of the AOS products were verified by GC/MS analysis after derivatization with methoxyamin and additionally in case of the □-ketols after silylation [22′].

### Biochemical Properties and Kinetic Parameters of Detergent-Free CYP74A 1

DNA sequence analysis of the clone pDEST14AOS5 revealed that the predicted amino acid sequence of the protein expressed from the clone was identical to that of the original cDNA clone, except that the N-terminal sequence, after removal of the chloroplastic transit peptide, was MASGSETPDLTVATRTG. Extraction of soluble His-tagged CYP74A1 activity required the presence of detergent, and only negligible activity was detected in its absence (data not shown). This suggested that CYP74A1 was associated with membranes in E. coli. The purified detergent-free CYP74A1 preparation was homogeneous as judged by SDS-PAGE (FIG. 7). Elution with histidine rather than imidazole, which is known to be a potent inhibitor of AOS [23′], was essential to produce an active enzyme with the characteristic UV/Visible spectrum shown in FIG. 7; the spectrum was not dependent on protein concentration. The protein had a Soret band at 391 nm, but a significant shoulder at 413 nm and major broad features at approximately 518, 545, 575 and 644 nm. The RZ of the purified protein was 1.1, which was very similar to that of CYP74C3 [15′] and suggested very little or no loss of haem. The molar extinction coefficient at 391 nm was calculated to be 89000±7000 M⁻¹.cm⁻¹.

A comparison of the kinetic parameters of detergent-free CYP74A1 with the substrates 13-HPOD/TE and 9-HPOD/TE (Table 1′) shows that although the k_{cat} with all four substrates were very similar, the differences in Kₘ meant that the catalytic efficiency (k_{cat}/Kₘ) with the different substrates was variable, but 13-HPOTE was the preferred substrate. The k_{cat}/Kₘ with 9-HPOTE, the least preferred substrate, was only 25% of that with 13-HPOTE. The k_{cat}/Kₘ with 13-HPODE and 9-HPODE was very similar at about 70% of that with 13-HPOTE.

HPLC/MS analysis confirmed the production of typical 9- and 13-AOS products in reactions of CYP74A1 (detergent-free and after reactivation with 50 μM Emulphogene micelle) with 9-HPOTE (FIG. 8a), 9-HPODE (FIG. 8b) and 13-HPOD/TE (data not shown). These included α-ketol and γ-ketol with m/z values of 309 (substrate: 13/9-HPOTE) and 311 (substrate: 13/9-HPODE) and of cyclopentenone with m/z values of 291 (13/9-HPOTE) and 293 (13/9-HPODE). The products in reactions of CYP74A1 with 9-HPODE are confirmed as 9-hydroxy-10-oxo-12-octadecenoic acid (α-ketol), 10-oxo-13-hydroxy-11-octadecenoic acid (γ-ketol) and the cyclopentenone 10-oxo-11-phytoenoic acid (10-OPEA). Reaction products with 9-HPOTE are similarly confirmed as 9-hydroxy-10-oxo-12-15-octadecadienoic acid (α-ketol), 10-oxo-13-hydroxy-11-15-octadecadienoic acid (γ-ketol) and the cyclopentenone 10-oxo-11-15-phytodienoic acid (10-OPDA).

Reactivation Kinetics with Detergent for CYP74A1

Detergent-free CYP74A1 was fully reactivated with 50 μM Emulphogene (FIG. 9). No reactivation was observed with 1 μM Emulphogene, which was slightly below the c.m.c. (1.25 μM). The K_{d} of Emulphogene micelle to CYP74A1 was estimated as 10.7±1.7 μM (FIG. 9). With 13-HPOTE and 13-HPODE as substrates, the k_{cat} of CYP74A1 after reactivation was increased 160- and 54-fold, respectively, than detergent-free enzyme (Table 1′). This effect was associated with an approximately 3- and 6-fold decrease in affinity for 13-HPOTE and 13-HPODE respectively. The k_{cat}/Kₘ of detergent-free CYP74A1 with 13-HPOTE and 13-HPODE was increased 50- and 10-fold respectively after reactivation with 5 mM Emulphogene. The k_{cat}/Kₘ of reactivated CYP74A1 with 13-HPODE was only 14% of that with 13-HPOTE, considerably lower than the corresponding figure of 75% for detergent-free protein. The kinetic parameters for CYP74A1 with 9-HPODE and 9-HPOTE were indistinguishable (Table 1′). The k_{cat} with both these substrates of only 2% of that with the preferred substrate 13-HPOTE, coupled with a 6-fold increase in Kₘ, meant that the k_{cat}/Kₘ of reactivated CYP74A1 with 9-HPODE or 9-HPOTE was negligible, at only 0.3% of that with the preferred substrate 13-HPOTE.

### Effects of Detergent on the Spin State Equilibrium of the Haem-Iron for CYP74A1

Changes in the UV/Visible spectrum were detected after reactivation of detergent-free CYP74A1 with Emulphogene. Addition of 50 μM Emulphogene to the detergent-free enzyme at 8.1 μM resulted in a change in the Soret region; the shoulder at 413 nm disappeared, an isosbestic point could be detected at 401 nm, and the absorbance at 391 nm increased, suggesting a shift in equilibrium towards a high-spin state of the haem iron. Similar changes at longer wavelengths were observed; the feature at 575 nm was slightly reduced, and the features at 518 and 644 nm were slightly increased and isosbestic points at 460, 523 and 593 nm were detected. EPR spectroscopy essentially confirmed the results from UV/Vis spectroscopy. The resting enzyme contained a mixture of high and low spin ferric haem iron with g factors of 8.07, 3.51, 1.66 and 2.39, 2.24, 1.93, respectively, in the ratio of 1:7. The relative concentrations of high and low spin haem iron were determined by comparison of the first integral of the low field (g 8.07) peak of the high spin ferric species and the first integral of the high field (g 1.93) peak of the low spin ferric species with the double integral of the copper standard spectrum using the method of Aasa and Vanngard [18′]. The addition of 1 mM 13-HPOTE to resting enzyme (100 μM) resulted in almost a complete loss of high spin haem iron with a corresponding increase in the proportion of low spin haem iron. Overall, there was no change in total EPR concentration, suggesting a shift from high to low spin state of the haem iron during turnover with substrate. Addition of 50 μM Emulphogene, well above the c.m.c. (1.25 μM), to resting enzyme (100 μM) resulted in partial conversion from high to low spin, with the ratio of high to low spin haem of 1:9.4, and no overall change in EPR concentration. The UV/Visible spectra and specific activities of the resting and detergent-reactivated enzymes after EPR were indistinguishable from those before the analysis.

### Oligomeric State of Detergent-Free CYP74A1

The molecular mass of His-tagged CYP74A1, based on the protein sequence and predicted from the cDNA, was 55.3 kDa. This was consistent with the value determined for the native protein by gel filtration (relative to globular protein standards) of molecular mass 47.2 kDa (FIG. 10). These measurements indicated that detergent-free CYP74A1 was a monomer. Almost no protein eluting at the molecular mass of a dimer, which, if present, would have been cleanly separated from monomer, could be detected by gel filtration, and only much smaller amounts of higher oligomers (larger than trimers) were present (FIG. 10). All the detergent-free fractions had negligible activity, compared to that of CYP74A1 after reactivation with 5 mM Emulphogene (FIG. 10).

### Oligomeric State of CYP74A1 in the Presence of Detergent

Gel filtration analysis of CYP74A1 in detergent-buffer containing 15.6 μM Emulphogene [15′], well above the c.m.c. (1.25 μM), indicated that it formed almost exclusively a species of molecular mass ˜110 kDa, as judged from the peak position on a calibrated Superdex 200 16/60 column and almost all activity was found at this peak position (FIG. 10). Only trace amounts of protein and activity were detected at peak positions corresponding to the size of a trimer, tetramer or higher oligomers.

Allene oxide synthase (AOS) from Arabidopsis thaliana (CYP74A1) was expressed in E. coli as an active protein with a C-terminal 4×His-tag, like coral AOS which also had a C-terminal 4×His-tag [24′] and similar to that described for tomato AOS with a C-terminal 8×His-tag [11′]. The protein lacked the N-terminal 32 amino acids that were identified as the chloroplastic transit peptide but, nevertheless, remained associated with membranes. CYP74A1 was expressed, without the N-terminal 21 amino acids, in E. coli in another study [1′], and this protein was also associated with membranes. These data together suggest that a sequence other than the N-terminal 32 amino acids acts as a membrane-binding domain. Despite the differences in sequence, CYP74A1 studied in [1′] and in the present work are kinetically very similar, exhibiting highest levels of activity with 13-HPOTE, and lower activities with 13-HPODE. We show for the first time, however, that CYP74A1 has 9-AOS activity in addition to the 13-AOS previously reported and indicates that, like a recombinant tomato AOS [11′], it exhibits dual specificity. The products detected in reactions of CYP74A1 with 9-HPODE correspond exactly to those detected using GC-MS in similar reactions with recombinant tomato AOS [11′]. CYP74A1 expressed in [1′] was not purified to homogeneity, and the oligomeric state was not reported. Gel filtration analysis has now confirmed that detergent-free CYP74A1 forms almost exclusively a highly water soluble monomeric protein, which is similar to the properties described for AOSs purified from flax seed [6′] and guayule rubber particles [7′] and for a recombinant coral AOS [24′]. All these proteins, like CYP74A1 in the present work, still required detergents for solubilisation and extraction. The effects of either the removal or addition of detergents on the oligomeric state of these proteins were, however, not investigated. Gel filtration analysis of homogeneously purified flax seed AOS in the presence of detergent micelles, at least, indicated a molecular mass of 55 kDa, which suggests that this protein is an entirely water soluble monomer and does not form any association with detergent micelles [6′]. In contrast, in the presence of detergent micelles, CYP74A1 in the present work formed a complex of 111.0 kDa, which corresponds almost exactly to the molecular mass of a protein dimer. The absence of protein dimers in concentrated solutions of detergent-free CYP74A1, and the fact that the average molecular mass of an Emulphogene micelle has been reported to be 62.0 kDa [15′] would, however, suggest that this complex was more likely an association between a protein monomer (47.2 kDa by gel filtration analysis) and a single detergent micelle (62.0 kDa).

The effects of the removal or addition of detergents on the activity of CYP74A enzymes in relation to oligomeric state have not been investigated. Detergents have been reported to increase the specific activity of AOS purified from flax seed [6′], but the mechanism for this activation and its relationship to oligomeric state have not been discussed. In the present work, detergent-free CYP74A1 had low, but significant, catalytic activity. The addition of detergent micelles, but not detergent monomers, to detergent-free CYP74A1, under the same conditions that induced the formation of a monomer-micelle complex, also caused a 48-fold increase in k_{cat}/Kₘ. The k_{cat}/Kₘ of up to 5.9×10⁷ M⁻¹.s⁻¹ with the preferred substrate 13-HPOTE is amongst the highest recorded. More recent work has also indicated that the most active species of CYP74C3 in the presence of detergent micelles is a monomer-micelle complex and that the detergent-free monomer can similarly be reactivated by detergent micelles [15′]; indeed, it was 2.6-fold faster than that reported for CYP74A1 in the present work. The behaviours of CYP74C3 and CYP74A1 in the presence of detergent micelles are, however, quite different; CYP74C3 had a tendency, in the presence of micelles, to form a much broader range of higher oligomers that were also highly active, whereas CYP74A1 formed almost exclusively a monomer-micelle complex, with only negligible amounts of protein forming higher oligomers. Highly concentrated and monodispersed samples of detergent-free CYP74A1 protein may, therefore, be preferable for the purposes of crystallisation and structural resolution of the first plant cytochrome P450 enzyme. This mechanism of activation, apparently unrelated to changes in oligomeric state of the protein, is unusual for cytochrome P450 enzymes. Taken together, the similar observations reported for both CYP74C3 and CYP74A1 may suggest that this is a common feature of members of the CYP74 sub-family of P450 enzymes. Although turnover with substrate for both enzymes was associated with a shift from high to low spin haem iron, the formation of a monomer-micelle complex in the absence of substrate for CYP74C3 and CYP74A1 was associated with a shift in equilibrium towards low and high spin haem iron, respectively. This would suggest that although the micelle bound the two enzymes with similar affinities (K_{d} 6.9 and 10.7 μM for CYP74C3 and CYP74A1, respectively), the conformational change around the haem that resulted was different and may relate to differences in hydrophobicity in the haem environments of the two proteins. One significant difference between CYP74C3 and CYP74A1 was the effect of micelle binding on the subsequent affinity of the complex for the preferred substrate 13-HPOTE. The k_{cat} of the monomer-micelle complex of CYP74C3 was 8-10 fold higher, and the Kₘ 5-6 fold lower, than the detergent-free monomer [15′]. In contrast, the k_{cat} of the monomer-micelle complex of CYP74A1 was 160-fold higher, and the Kₘ was also 3-fold higher, than the detergent-free monomer. Despite these differences, however, there was a ˜50-fold increase in k_{cat}/Kₘ for both enzymes upon micelle binding.

A comparison of the k_{cat}/Kₘ—the most rigorous measure of substrate specificity—of CYP74A1 before and after reactivation with an Emulphogene micelle clearly demonstrates that the substrate specificity of CYP74A1 was modified upon micelle-binding. The large changes in the kinetic parameters of CYP74A1 would suggest that the conformational change that was induced in the detergent-free protein upon micelle-binding affected the interaction with substrates. That this change was in the haem domain of the protein, and most likely where the substrates are bound, was supported by the similar changes detected by EPR spectroscopy in the spin state equilibrium of the haem iron upon micelle-binding. This contrasts with CYP74C3 where, despite similar micelle-induced changes in the spin state equilibrium of the haem iron, and a similar K_{d} for micelle-binding, the substrate specificity of detergent-free CYP74C3 was indistinguishable from that after reactivation with detergent micelle [15′].

Compartmentalisation of the substrates and enzymes of oxylipin metabolism in the plant cell is a key control mechanism that determines the oxylipin profile of a plant [2′]. The new observation in the present work of the capacity for 9-AOS activity in Arabidopsis was unexpected but clearly of relevance to its biological activity in vivo, as is the tendency of CYP74A1 to form a strong micelle association. Although there is no example of a plant AOS, except for the rubber particle protein [7′], that is not targeted to the chloroplast under natural conditions, the consequences of a change in AOS localisation have, however, been demonstrated in transgenic plants that were over-expressing AOS [25′]; localisation of the transgenic AOS protein in the cytosol directly effected changes in both the oxylipin profile of the transgenic plants and the response to a pest or pathogen. Although we have used an in vitro system for studying CYP74A1, detergent micelles essentially represent an artificial membrane and the chloroplast envelope contains a lot of phospholipids [26′] that could potentially interact with AOS. We suggest that the in vivo biological activity of AOS, at least in Arabidopsis, may be dependent on whether the AOS protein remains associated with the chloroplast membrane, or becomes dissociated and water-soluble, either in the intermembrane space of the chloroplast or in the cytosol. The final location of any membrane-dissociated AOS may well be dependent on the specificity of that AOS. Thus, recent evidence of a 9-AOS from potato that was localized to the outer membrane of the chloroplast, and apparently not imported into the chloroplast [22′], is consistent with the production of 9-LOX products in the cytosol [2′] and suggests that dissociated 9-AOS would be cytosolic. In contrast, 13-AOS has been localized to the inner membrane of the chloroplast [14′] such that dissociated 13-AOS would reside inside the chloroplast or possibly in the intermembrane space. The situation may be further complicated in Arabidopsis since CYP74A1 exhibits dual specificity. All these factors may change quite considerably, for example, during an attack by a pest or pathogen where a number of different extracellular enzymes [27′] or effector proteins [28′] may be secreted that are potentially able, directly or indirectly, to release AOS from its membrane-bound state. The evidence from the present work would suggest that if this were indeed the case, in addition to the likely changes in substrate availability as a result of the pest or pathogen interaction, there would also be a dramatic reduction in AOS activity and a change in AOS substrate specificity that would most likely affect the oxylipin profile of the plant. Our recent work showing similar behaviour in vitro for a hydroperoxide lyase (HPL) from Medicago truncatula [15′] would suggest that some of these considerations afforded to AOS should be given also to HPL. It remains to be determined if the effects observed in our work with AOS and HPL in vitro can account for some of the observations to be made in planta.

TABLE 1′
Kinetic parameters of CYP74A1: effect of detergent

| Kinetic parameter | Detergent-free | Reactivated † |
|---|---|---|
| k_{cat} (s⁻¹) * | | |
| 13-HPOTE | 3.3 ± 0.1 | 528 ± 15 |
| 13-HPODE | 4.2 ± 0.2 | 226 ± 9 |
| 9-HPOTE | 3.2 ± 0.1 | 11 ± 1 |
| 9-HPODE | 4.9 ± 0.4 | 12 ± 1 |
| Kₘ (μM) | | |
| 13-HPOTE | 2.8 ± 0.5 | 9.3 ± 1.0 |
| 13-HPODE | 4.8 ± 0.7 | 28.0 ± 4.0 |
| 9-HPOTE | 10.5 ± 1.5 | 58.4 ± 7.9 |
| 9-HPODE | 5.9 ± 1.6 | 57.5 ± 7.4 |
| k_{cat}/Kₘ (μM⁻¹ · s⁻¹) | | |
| 13-HPOTE | 1.2 | 58.7 |
| 13-HPODE | 0.9 | 8.1 |
| 9-HPOTE | 0.3 | 0.2 |
| 9-HPODE | 0.8 | 0.2 |
| Substrate specificity | | |
| (% 13-HPOTE) | | |
| 13-HPOTE | 100 | 100 |
| 13-HPODE | 75 | 14 |
| 9-HPOTE | 25 | 0.3 |
| 9-HPODE | 67 | 0.3 |

* Assuming 1 active site per monomer of molecular mass 55.3 kDa
† With 50 μM Emulphogene micelle
Error bars are the means ± standard errors of at least five determinations on two different enzyme preparations

### Example 3

### Stability of Dried Detergent-Free HPL and AOS

The following table provides a summary enzyme stability studies conducted on HPL and AOS enzymes, subjected to either freeze drying or speedvac drying, in the presence or absence of detergent. What is reported here is the degree to which enzyme activity is lost based on the storage condition. As can be seen, for HPLF, AOS and Pepper HPL, in each case, the lowest amount of active protein loss occurs when the enzyme is dried in a detergent free composition. Subsequent re-hydration of each dried enzyme and spectroscopic analysis was used to determine the stability of the thus-treated enzymes. FIG. 11 provides stability over time information for the thus stored enzymes showing stability for at least ten weeks.

UV/Vis spectra of detergent-free preparations of CYP74C3 (HPLF), CYP74A1 (AOS) and Pepper HPL were determined before and after freeze drying, as well as before and after drying in a speedvac. In addition, the UV/Vis spectra of preparations of CYP74C3 (HPLF), CYP74A1 (AOS) and Pepper HPL in emulphogene detergent before and after freeze drying, as well as before and after drying in a speedvac, were determined. The UV/Vis spectra of preparations of CYP74C3 (HPLF), CYP74A1 (AOS) and Pepper HPL in the detergent triton before and after freeze drying were further determined. The UV/Vis spectra of these preparations were used to determine the percentage loss of protein resulting from freeze drying, or drying in a speedvac and the results are summarised in the following table.

The stability of freeze dried preparations of CYP74C3 (HPLF) and CYP74A1 (AOS) over 15 weeks is shown in FIG. 12.

| | Loss of active protein (%)* | | |
|---|---|---|---|
| Treatment | HPLF | AOS | Pepper HPL |
| Freeze Drying | | | |
| Detergent-free | 5.1 | 2.3 | 9.3 |
| +Emulphogene (0.3% w/v) | 73.2 | 58.1 | 89.6 |
| +Triton X-100 (0.2% w/v) | 59.0 | 41.3 | 74.4 |
| Speedvac Drying | | | |
| Detergent-free | 23.1 | 17.6 | 1.3 |
| +Emulphogene (0.3% w/v) | 63.2 | 83.1 | 78.8 |

*Calculated from haem absorption at 391 nm

### Example 4

### Stability of HPL and AOS Stored in a Dried Detergent Free Composition as Compared with the Same Enzymes Stored in Detergent Free or Detergent Containing Solution at 4 Degrees and Minus Twenty Degrees Centigrade

Stability tests were conducted on dried (detergent free) HPLF/AtAOS reconstituted with phosphate buffer containing 5 mM Emulphogene, compared with stability of alfalfa HPL stored at 4° C. with Triton X-100, stability of HPLF and AOS stored at 4° C. or −20° C. with Emulphogene plus 20% glycerol to inhibit freezing.

Comparison of FIG. 12, (stability of CYP74 enzymes stored dried in the absence of detergent then reconstituted with a detergent-containing buffer and tested), with FIGS. 13, 14, 15, 16 (storage of CYP74 enzymes in solution with or without detergent, at either 4 or −20 degrees Centigrade), demonstrates very clearly that the CYP74 enzymes retain essentially 100% of their activity over an extended period of time, up to 15 weeks at a minimum, when stored dried in the absence of detergent, as compared to very rapid drop-off in activity when stored in solution, with or without detergent, at either 4 degrees or −20 degrees Centigrade.

We anticipate these results to apply to all members of the CYP74 enzymes. These results are related to AOS (CYP74A and C) and HPL (CYP74B and C). DES, a CYP74D, is expected to behave similarly.

### REFERENCES FOR CYP74C3

1. Schoch, G. A., Attias, R., Belghazi, M., Dansette, P. M. and Werck-Reichhart, D. (2003) Engineering of a water-soluble plant cytochrome P450, CYP73A1, and NMR-based orientation of natural and alternate substrates in the active site. Plant Physiol. 133, 1198-1208
2. Hasemann, C. A., Kurumbail, R. G., Boddupalli, S. S., Peterson, J. A. and Desienhofer, J. (1995) Structure and function of cytochromes P450: a comparative analysis of three crystal structures. Structure 2, 41-62
3. Williams, P. A., Cosme, J., Sridhar, V., Johnson, E. F. and McRee, D. E. (2000) Mammalian microsomal cytochrome P450 monooxygenase: structural adaptations for membrane binding and functional diversity. Mol. Cell. 5, 121-131
4. Song, W-C, Funk, C. D. and Brash, A. R. (1993) Molecular cloning of an allene oxide synthase: a cytochrome P450 specialised for the metabolism of fatty acid hydroperoxides. Proc. Natl. Acad. Sci. U.S.A. 90, 8519-8523
5. Wang, L.-H., Tsai, A-L. and Hsu, P-Y. (2001) Substrate binding is the rate-limiting step in thromboxane synthase catalysis. J. Biol. Chem. 276, 14737-14743
6. Grechkin, A. N., Mukhtarova, L. S, and Hamberg, M. (2003) Detection of an enol intermediate in the hydroperoxide lyase chain cleavage reaction. FEBS Lett. 549, 31-34
7. Casey, R. and Hughes, R. K. (2004) Recombinant lipoxygenases and oxylipin metabolism in relation to food quality. Food Biotechnol. 18, 135-170
8. Morant, M., Bak, S., Moller, B. L. and Werck-Reichhart, D. (2003) Plant cytochromes P450: tools for pharmacology, plant protection and phytoremediation. Curr. Opin. Biotechnol. 14, 151-162
9. Oldham, M. L., Brash, A. R. and Newcomer, M. E. (2005) The structure of coral allene oxide synthase reveals a catalase adapted for metabolism of a fatty acid hydroperoxide. Proc. Natl. Acad. Sci. U.S.A. 102, 297-302
10. Pan, Z., Camara, B., Gardner, H. W. and Backhaus, R. A. (1998) Aspirin inhibition and acetylation of the plant cytochrome P450, allene oxide synthase, resembles that of animal prostaglandin endoperoxide H synthase. J. Biol. Chem. 273, 18139-18145
11. Mast, N., Andersson, U., Nakayama, K., Bjorkhem, I. and Pikulevaa, I. A. (2004) Expression of human cytochrome P450 46A1 in Escherichia coli: effects of N- and C-terminal modifications. Arch. Biochem. Biophys. 428, 99-108
12. von Wachenfeldt, C., Richardson, T. H., Cosme, J. and Johnson, E. F. (1997) Microsomal P450 2C3 is expressed as a soluble dimer in Escherichia coli following modification of its N-terminus. Arch. Biochem. Biophys. 339, 107-114
13. Noordermeer, M. A., van Dijken, A. J. H., Smeekens, S. C. M., Veldink, G. A. and Vliegenthart, J. F. G. (2000) Characterisation of three cloned and expressed 13-hydroperoxide lyase isoenzymes from alfalfa with unusual N-terminal sequences and different enzyme kinetics. Eur. J. Biochem. 267, 2473-2482
14. Dean, W. L. and Gray, R. D. (1982) Relationship between state of aggregation and catalytic activity for cytochrome P-450LM2 and NADPH-cytochrome P-450 reductase. J. Biol. Chem. 257, 14679-14685
15. Tijet, N., Waspi, U., Gaskin, D. J., Hunziker, P., Muller, B. L, Vulfson, E. N., Slusarenko, A., Brash, A. R. and Whitehead, I. M. (2000) Purification, molecular cloning, and expression of the gene encoding fatty acid 13-hydroperoxide lyase from guava fruit (Psidium guajava). Lipids 35, 709-720
16. Shibata, Y., Matsui, K., Kajiwara, T. and Hatanaka, A (1995) Purification and properties of fatty-acid hydroperoxide lyase from green bell pepper fruits. Plant Cell Physiol. 36, 147-156
17. Husson, F. and Belin, J. M. (2002) Purification of hydroperoxide lyase from green bell pepper (Capsicum annuum L.) fruits for the generation of C6-aldehydes in vitro. J. Agric. Food Chem. 50, 1991-1995
18. Itoh, A. and Vick, B. A. (1999) The purification and characterisation of fatty acid hydroperoxide lyase in sunflower. Biochim. Biophys. Acta 1436, 531-540
19. Schreier, P. and Lorenz, G. (1982) Separation, partial-purification and characterisation of a fatty-acid hydroperoxide cleaving enzyme from apple and tomato fruits. Zeitschrift. Naturforsch. 37, 165-173
20. Fauconnier, M-L., Perez, A. G., Sanz, C. and Marlier, M. (1997) Purification and characterisation of tomato leaf (Lycopersicon esculentum Mill.) hydroperoxide lyase. J. Agric. Food Chem. 45, 4232-4236
21. Matsui, K., Miyahara, C., Wilkinson, J., Hiatt, B., Knauf, V. and Kajiwara, T. (2000) Fatty acid hydroperoxide lyase in tomato fruits: cloning and properties of a recombinant enzyme expressed in Escherichia coli. Biosci. Biotechnol. Biochem. 64, 1189-1196
22. Olias, J. M., Rios, J. L., Valle, M., Zamora, R., Sartz, L. C. and Axelrod, B. (1990) Fatty-acid hydroperoxide lyase in germinating soybean seedlings. J. Agric. Food Chem. 38, 624-630
23. Vick, B. A. and Zimmerman, D. C. (1976) Lipoxygenase and hydroperoxide lyase in germinating watermelon seedlings. Plant Physiol. 57, 780-788
24. Psylinakis, E., Davoras, E. M., Ioannidis, N., Trikeriotis, M., Petrouleas, V. and Ghanotakis, D. F. (2001) Isolation and spectroscopic characterisation of a recombinant bell pepper hydroperoxide lyase. Biochim. Biophys. Acta 1533, 119-127
25. Kandzia, R., Stumpe, M., Berndt, E., Szalata, M., Matsui, K. and Feussner, I. (2003) On the specificity of lipid hydroperoxide fragmentation by fatty acid hydroperoxide lyase from Arabidopsis thaliana. J. Plant Physiol. 160, 803-809
26. Howe, G. A., Lee, G. I., Itoh, A., Li, L., DeRocher, A. E. (2000) Cytochrome P450-dependent metabolism of oxylipins in tomato. Cloning and expression of allene oxide synthase and fatty acid hydroperoxide lyase. Plant Physiol 123, 711-724
27. Matsui, K., Ujita, C., Fujimoto, S-H., Wilkinson, J., Hiatt, B., Knauf, V., Kajiwara, T. and Feussner, I. (2000) Fatty acid 9- and 13-hydroperoxide lyases from cucumber. FEBS Lett. 481, 183-188
28. Tijet, N., Schneider, C., Muller, B. L. and Brash, A. R. (2001) Biogenesis of volatile aldehydes from fatty acid hydroperoxides: molecular cloning of a hydroperoxide lyase (CYP74C) with specificity for both the 9- and 13-hydroperoxides of linoleic and linolenic acids. Arch. Biochem. Biophys. 386, 281-289
29. Koeduka, T., Stumpe, M., Matsui, K., Kajiwara, T. and Feussner, I. (2003) Kinetics of barley FA hydroperoxide lyase are modulated by salts and detergents. Lipids 38, 1167-1172
30. Scott, E. E., He, Y. A., Wester, M. R., White, M. A., Chin, C. C., Halpert, J. R., Johnson, E. F. and Stout, C. D. (2003) An open conformation of mammalian cytochrome P450 2B4 at 1.6-Å resolution. Proc. Natl. Acad. Sci. USA 100, 13196-13201
31. Schoch, G. A., Yano, J. K., Wester, M. R., Griffin, C., Stout, E. F. and Johnson, E. F. (2004) Structure of human microsomal cytochrome P450 2C8: evidence for a peripheral fatty acid binding site. J. Biol. Chem. 279, 9497-9503
32. Williams, P. A., Cosme, J., Ward, A., Angove, H. C., Vinkovic, D. M. and Jhoti, H. (2003) Crystal structure of human cytochrome P450 2C9 with bound warfarin. Nature 424, 464-468
33. Yano, J. K., Wester, M. R., Schoch, G. A., Griffin, K. J., Stout, C. D. and Johnson, E. F. (2004) The structure of human microsomal cytochrome P450 3A4 determined by X-ray crystallography to 2.05-A resolution. J. Biol. Chem. 279, 38091-38094
34. Yano, J. K., Hsu, M. H., Griffin, K. J., Stout, C. D., Johnson, E. F. (2005) Structures of human microsomal cytochrome P450 2A6 complexed with coumarin and methoxsalen. Nat. Struct. Mol. Biol. 12, 822-823
35. Zhukovsky, E. A., Lee, J. O., Villegas, M., Chan, C., Chu, S, and Mroske, C. (2004) TNF ligands: Is TALL-1a trimer or a virus-like cluster? Nature 427, 413-414
36. Vick, B. A. (1991) A spectrophotometric assay for hydroperoxide lyase. Lipids 26, 315-320
37. Aase, R. and Vanngard, T. (1975) EPR signal intensity and powder shapes—re-examination. J. Magn. Res. 19, 308-315
38. Berry, E. A. and Trumpower, B. (1987) Simultaneous determination of haems a, b, and c from pyridine haemochrome spectra. Anal. Biochem. 161, 1-15
39. Bradford, M. M. (1976) A rapid and sensitive method for the quantitation of microgram quantities of protein utilizing the principle of protein-dye binding. Anal. Biochem. 72, 248-254
40. Helenius, A. and Simons, K. (1975) Solubilisation of membranes by detergents. Biochim. Biophys. Acta 415, 29-79
41. Noordermeer, M. A., Veldink, G. A. and Vliegenthart, J. F. G. (1999) Alfalfa contains substantial 9-hydroperoxide lyase activity and a 3Z:2E-enal isomerase. FEBS Lett. 443, 201-204
42. Schuck, P. (2000) Size-distribution analysis of macromolecules by sedimentation velocity ultracentrifugation and lamm equation modeling. Biophys. J. 78, 1606-1619
43. Colfen, H. and Harding, S. E. (1997) MSTARA and MSTARI: Interactive PC algorithms for simple, model independent evaluation of sedimentation equilibrium data. Eur. Biophys. J. Biophys. Lett. 24, 333-346
44. Laue, T. M., Shah, B. D., Ridgeway, T. M. and Pelletier, S. L. (1992) Analytical Ultracentrifugation in Biochemistry and Polymer Science (Harding, S. E., Rowe, A. J. and Horton, J. C. eds.), pp. 90-125, Royal Society of Chemistry, Cambridge, U.K.
45. Squire, P. G. and Himmel, M. E. (1979) Hydrodynamics and protein hydration. Arch. Biochem. Biophys. 196, 165-177
46. Rahfeld, J-U., Rucknagel, P., Stoller, G., Horne, S. M., Schierhorn, A., Young, K. D. and Fischer, G. (1996) Isolation and amino acid sequence of a new 22-kDa FKBP-like peptidyl-prolyl cis/trans-isomerase of Escherichia coli: similarity to Mip-like proteins of pathogenic bacteria. J. Biol. Chem. 271, 22130-22138
47. Abraham, B. D., Sono, M., Boutaud, O., Shriner, A., Dawson, J. H., Brash, A. R. and Gaffney, B. J. (2001) Characterisation of the coral allene oxide synthase active site with UV-visible absorption, magnetic circular dichroism, and electron paramagnetic resonance spectroscopy: evidence for tyrosinate ligation to the ferric enzyme haem iron. Biochemistry 40, 2251-2259
48. Song, W-C. and Brash, A. R. (1991) Purification of an allene oxide synthase and identification of the enzyme as a cytochrome P-450. Science 253, 781-784
49. Noordermeer, M. A., Veldink, G. A. and Vliegenthart, J. F. G. (2001) Spectroscopic studies on the active site of hydroperoxide lyase: the influence of detergents on its conformation. FEBS Lett. 489, 229-232
50. White, R. E. and Coon, M. J. (1982) Haem ligand replacement reactions of cytochrome P-450. Characterisation of the bonding atom of the axial ligand trans to thiolate as oxygen. J. Biol. Chem. 257, 3073-3083
51. Rodriguez-Lopez, J. N., Lowe, D. J., Hernandez-Ruiz, J., Hiner, A. N. P., Garcia-Canovas, F. and Thorneley, R. N. F. (2001) Mechanism of reaction of hydrogen peroxide with horseradish peroxidase: identification of intermediates in the catalytic cycle. J. Am. Chem. Soc. 123, 11838-11847
52. Lindskog, S., Engberg, P., Forsman, C., Ibrahim, S. A., Johnson, B. H., Simosson, I. and Tibell, L. (1984) Kinetics and mechanism of carbonic anhydrase isoenzymes. Ann. N.Y. Acad. Sci. 429, 61-75
53. Haugen, D. A. and Coon, M. J. (1976) Properties of electrophoretically homogeneous phenobarbital-inducible and beta-naphthoflavone-inducible forms of liver microsomal cytochrome P-450. J. Biol. Chem. 251, 7929-7939
54. Wagner, S. L. and Gray, R. D. (1985) Effects of detergent on substrate binding and spin state of purified liver microsomal cytochrome P-450LM2 from phenobarbital-treated rabbits. Biochemistry 24, 3809-3814
55. Viner, R. I, Novikov, K. N., Ritov, V. B., Kagan, V. E. and Alternan, M. A. (1995) Effect of different solubilizing agents on the aggregation state and catalytic activity of two purified rabbit cytochrome P450 isozymes, CYP1A2 (LM4) and CYP2B4 (LM2). Biochem. Biophys. Res. Comm. 217, 886-891

### REFERENCES FOR CYP74A1

[1′] Laudert, D. L., Pfannschmidt, U., Lottspeich, F., Hollander-Czytko, H. and Weiler, E. W. (1996) Cloning, molecular and functional characterisation of Arabidopsis thaliana allene oxide synthase (CYP74), the first enzyme of the octadecanoid pathway to jasmonates. Plant Mol. Biol. 31, 323-335.
[2′] Feussner, I. and Wasternack, C. (2002) The lipoxygenase pathway. Ann. Rev. Plant Physiol. Plant Mol. Biol. 53, 275-297
[3′] Song, W-C, Funk, C. D. and Brash, A. R. (1993) Molecular cloning of an allene oxide synthase: a cytochrome P450 specialised for the metabolism of fatty acid hydroperoxides. Proc. Natl. Acad. Sci. U.S.A. 90, 8519-8523.
[4′] Veldink, G. A., Vliegenthart, J. F. G. and Boldingh, J. (1970) The enzymic conversion of linoleic acid hydroperoxide by flax-seed hydroperoxide isomerase. Biochem. J. 120, 55-60.
[5′] Pan, Z., Camara, B., Gardner, H. W. and Backhaus, R. A. (1998) Aspirin inhibition and acetylation of the plant cytochrome P450, allene oxide synthase, resembles that of animal prostaglandin endoperoxide H synthase. J. Biol. Chem. 273, 18139-18145.
[6′] Song, W-C. and Brash, A. R. (1991) Purification of an allene oxide synthase and identification of the enzyme as a cytochrome P-450. Science 253, 781-784.
[7′] Pan, Z., Durst, F., Werck-Reichhart, D., Gardner, H. W., Camara, B., Cornish, K. and Backhaus, R. A. (1995) The major protein of guayule rubber particles is a cytochrome P450. J. Biol. Chem. 270, 8487-8494.
[8′] Utsunomiya, Y., Nakayama, T., Oohira, H., Hirota, R., Mori, T., Kawai, F. and Ueda, T. (2000) Purification and inactivation by substrate of an allene oxide synthase (CYP74) from corn (Zea mays L.) seeds. Phytochem. 53, 319-323.
[9′] Maucher, H., Hause, B., Feussner, I., Ziegler, J. and Wasternack, C. (2000) Allene oxide synthases of barley (Hordeum vulgare cv. Salome): tissue specific regulation in seedling development. Plant J. 21, 199-213.
[10′] Howe, G. A., Lee, G. I., Itoh, A., Li, L. and DeRocher, A. E. (2000) Cytochrome P450-dependent metabolism of oxylipins in tomato: cloning and expression of allene oxide synthase and fatty acid hydroperoxide lyase. Plant Physiol. 123, 711-724.
[11′] Itoh, A, Schilmiller, A. L., McCaig, B. C. and Howe, G. A. (2002) Identification of a jasmonate-regulated allene oxide synthase that metabolises 9-hydroperoxides of linoleic and linolenic acids. J. Biol. Chem. 277, 46051-46058.
[12′] Oldham, M. L., Brash, A. R. and Newcomer, M. E. (2005) The structure of coral allene oxide synthase reveals a catalase adapted for metabolism of a fatty acid hydroperoxide. Proc. Natl. Acad. Sci. U.S.A. 102, 297-302.
[13′] Koljak, R., Boutaud, O., Shieh, B-H., Samel, N. and Brash, A. R. (1997) Identification of a naturally occurring peroxidase-lipoxygenase fusion protein. Science 277, 1994-1996.
[14′] Froehlich, J. E., Itoh, A. and Howe. G. A. (2001) Tomato allene oxide synthase and fatty acid hydroperoxide lyase, two cytochrome P450s involved in oxylipin metabolism, are targeted to different membranes of chloroplast envelope. Plant Physiol. 125, 306-317.
[15′] Hughes, R. K., Belfield, E. J., Muthusamay, M., Khan, A., Rowe, A., Harding, S. E., Fairhurst, S. A., Bornemann, S., Ashton, R., Thorneley, R. N. F. and Casey, R. (2006) Characterisation of Medicago truncatula hydroperoxide lyase (CYP74C3), a water-soluble detergent-free cytochrome P450 monomer whose biological activity is defined by monomer-micelle association. Biochem. J. 395, 641-652.
[16′] Emanuelsson, O., Nielsen, H. and von Heijne, G. (1999) ChloroP, a neural network-based method for predicting chloroplast transit peptides and their cleavage sites. Prot. Sci. 8, 978-984.
[17′] Vick, B. A. (1991) A spectrophotometric assay for hydroperoxide lyase. Lipids 26, 315-320.
[18′] Aasa, R. and Vanngard, T. (1975) EPR signal intensity and powder shapes—reexamination. J. Magn. Res. 19, 308-315.
[19′] Berry, E. A. and Trumpower, B. (1987) Simultaneous determination of haems a, b, and c from pyridine haemochrome spectra. Anal. Biochem. 161, 1-15.
[20′] Bradford, M. M. (1976) A rapid and sensitive method for the quantitation of microgram protein utilizing the principle of protein-dye binding. Anal. Biochem. 72, 248-254.
[21′] Helenius, A. and Simons, K. (1975) Solubilisation of membranes by detergents. Biochim. Biophys. Acta 415, 29-79.
[22′] Stumpe, M., Göbel, C., Demchenko, K., Hoffmann, M., Klösgen, R. B., Pawlowski, K., Feussner, I. (2006) Identification of an allene oxide synthase (CYP74C) that leads to formation of α-ketols from 9-hydroperoxides of linoleic and linolenic acid in below ground organs of potato. Plant J. (in press).
[23′] Oh, K. and Murofushi, N. (2002) Design and synthesis of novel imidazole derivatives as potent inhibitors of allene oxide synthase (CYP74). Bioorg. Med. Chem. 10, 3707-3711.
[24′] Boutaud, O. and Brash, A. R. (1999) Purification and catalytic activities of the two domains of the allene oxide synthase-lipoxygenase fusion protein of the coral Plexaura homomalla. J. Biol. Chem. 274, 33764-33770.
[25′] Wang, C. X., Avdiushko, S, and Hildebrand, D. F. (1999) Over-expression of a cytoplasm-localised allene oxide synthase promotes the wound-induced accumulation of jasmonic acid in transgenic tobacco. Plant Mol. Biol. 40, 783-793.
[26′] Joyard, J., Teyssier, E., Miege, C., Berny-Seigneurin, D., Marechal, E., Block, M. A., Dorne, A. J., Rolland, N., Ajlani, G. and Douce, R. (1998) The biochemical machinery of plastid envelope membranes. Plant Physiol. 118, 715-723.
[27′] Smolka, M. B., Martins, D., Winck, F. V., Santoro, C. E., Castellari, R. R., Ferrari, F., Brum, I. J., Galembeck, E., Coletta, H. D., Machado, M. A., Marangoni, S, and Novello, J. C. (2003) Proteome analysis of the plant pathogen Xylella fastidiosa reveals major cellular and extracellular proteins and a peculiar codon bias distribution. Proteomics 2, 224-237.
[28′] Setubal, J. C., Moreira, L. M. and da Silva, A. C. R. (2005) Bacterial phytopathogens and genome science. Curr. Opin. Microbiol. 8, 595-600.

1. A method for producing, and optionally storing, a stable preparation of an isolated CYP74 enzyme which comprises the steps of:
(i) providing the enzyme in a substantially detergent-free state, and
(ii) drying the detergent free enzyme.

2. A method according to claim 1 wherein the CYP74 enzyme is provided by recombinant expression.

3. A method according to claim 1 wherein the CYP74 enzyme is provided in the presence of detergent which is subsequently removed to provide the enzyme in a substantially detergent-free state.

4. A method according to claim 1 wherein the drying is performed by a method selected from the list consisting of: freeze drying, spin-vacuum drying, thin film spray drying.

5. A method according to claim 1 which further comprises the step of:
(iii) storing the preparation.

6. A method according to claim 5 wherein the enzyme is stored at room temperature.

7. A method according to claim 5 wherein the enzyme is stored at around 4 degrees Centigrade.

8. A method according to claim 5 wherein the enzyme is stored at less than 0 degrees Centigrade.

9. A method according to claim 1 which further comprises the step of:
(iv) solubilising the preparation in a detergent containing solution, or in a solution to which a detergent is added, such as to provide active CYP74 enzyme

10. A method according to claim 9 wherein the active CYP74 enzyme exhibits HPL activity, AOS activity, or both.

11. A method according to claim 9 wherein the preparation is capable of being stored for at least 15 weeks while losing less than 25%, 20%, 15%, or 10% CYP74 enzyme activity.

12. A method according to claim 9 wherein the loss of activity in the preparation is less than 50%, 40%, 30%, 20%, or 10% of the corresponding loss activity of the corresponding CYP74 enzyme when stored in the presence of detergent under the same conditions for 15 weeks.

13. A method according to claim 1 wherein the preparation comprises two CYP74 enzymes.

14. A method according to claim 1 wherein the preparation comprises CYP74C3, CYP74A1, or both.

15. A stable preparation of an isolated CYP74 enzyme in dry form and substantially free of detergent, which is capable of being stored for at least 15 weeks while losing less than 25%, 20%, 15%, or 10% CYP74 enzyme activity when subsequently solubilised in a detergent containing solution, or in a solution to which a detergent is added.

16. A preparation according to claim 15 which is freeze dried; spin-vacuum dried, or thin film spray dried.

17. A preparation according to claim 15 which comprises an excipient.

18. A preparation according to claim 15 wherein the active CYP74 enzyme exhibits HPL activity, AOS activity, or both.

19. A preparation according to claim 15 wherein the preparation comprises two CYP74 enzymes.

20. A preparation according to claim 15 wherein the preparation comprises CYP74C3, CYP74A1, or both.

21. A method of converting hydroperoxy compounds to \acute{ω}-oxo acids, volatile aldehydes, or allene oxides which comprises:
(i) providing a preparation according to claim 15,
(ii) solubilising the preparation in a detergent containing solution, or in a solution to which a detergent is added,
(iii) contacting the hydroperoxy compound with the solubilised preparation,
such as to catalyze the conversion to the \acute{ω}-oxo acids, volatile aldehydes, or allene oxides.
